SEQUENCE LISTING (13 sequences)

SEQ ID NO: 1 - DNA, 4107 bp, Homo sapiens
gcgcaaccctccggaagctgccgcccctttccccttttatgggaatactttttttaaaaaaaaagagttcgctggcgccaccccgtaggactggccgccctaaaaccgtgataaaggagctgctcgccacttctcacttccgcttccttccagtaaggagtcggggtcttccccagttttctcagccaggcggcggcggcgactggcaatgtttggcctcaaaagaaacgcggtaatcggactcaacctctactgtgggggggccggcttgggggccggcagcggcggcgccacccgcccgggagggcgacttttggctacggagaaggaggcctcggcccggcgagagatagggggaggggaggccggcgcggtgattggcggaagcgccggcgcaagccccccgtccaccctcacgccagactcccggagggtcgcgcggccgccgcccattggcgccgaggtccccgacgtcaccgcgacccccgcgaggctgcttttcttcgcgcccacccgccgcgcggcgccgcttgaggagatggaagccccggccgctgacgccatcatgtcgcccgaagaggagctggacgggtacgagccggagcctctcgggaagcggccggctgtcctgccgctgctggagttggtcggggaatctggtaataacaccagtacggacgggtcactaccctcgacgccgccgccagcagaggaggaggaggacgagttgtaccggcagtcgctggagattatctctcggtaccttcgggagcaggccaccggcgccaaggacacaaagccaatgggcaggtctggggccaccagcaggaaggcgctggagaccttacgacgggttggggatggcgtgcagcgcaaccacgagacggccttccaaggcatgcttcggaaactggacatcaaaaacgaagacgatgtgaaatcgttgtctcgagtgatgatccatgttttcagcgacggcgtaacaaactggggcaggattgtgactctcatttcttttggtgcctttgtggctaaacacttgaagaccataaaccaagaaagctgcatcgaaccattagcagaaagtatcacagacgttctcgtaaggacaaaacgggactggctagttaaacaaagaggctgggatgggtttgtggagttcttccatgtagaggacctagaaggtggcatcaggaatgtgctgctggcttttgcaggtgttgctggagtaggagctggtttggcatatctaataagatagccttactgtaagtgcaatagttgacttttaaccaaccaccaccaccaccaaaaccagtttatgcagttggactccaagctgtaacttcctagagttgcaccctagcaacctagccagaaaagcaagtggcaagaggattatggctaacaagaataaatacatgggaagagtgctccccattgattgaagagtcactgtctgaaagaagcaaagttcagtttcagcaacaaacaaactttgtttgggaagctatggaggaggacttttagatttagtgaagatggtagggtggaaagacttaatttccttgttgagaacaggaaagtggccagtagccaggcaagtcatagaattgattacccgccgaattcattaatttactgtagtgttaagagaagcactaagaatgccagtgacctgtgtaaaagttacaagtaatagaactatgactgtaagcctcagtactgtacaagggaagcttttcctctctctaattagctttcccagtatacttcttagaaagtccaagtgttcaggacttttatacctgttatactttggcttggtttccatgattcttactttattagcctagtttatcaccaataatacttgacggaaggctcagtaattagttatgaatatggatatcctcaattcttaagacagcttgtaaatgtatttgtaaaaattgtatatatttttacagaaagtctatttctttgaaacgaaggaagtatcgaatttacattagtttttttcatacccttttgaactttgcaacttccgtaattaggaacctgtttcttacagcttttctatgctaaactttgttctgttcagttctagagtgtatacagaacgaattgatgtgtaactgtatgcagactggttgtagtggaacaaatctgataactatgcaggtttaaattttcttatctgattttggtaagtattccttagataggtttttctttgaaaacctgggattgagaggttgatgaatggaaattctttcacttcattatatgcaagttttcaataattaggtctaagtggagttttaaggttactgatgacttacaaataatgggctctgattgggcaatactcatttgagttccttccatttgacctaatttaactggtgaaatttaaagtgaattcatgggctcatctttaaagcttttactaaaagattttcagctgaatggaactcattagctgtgtgcatataaaaagatcacatcaggtggatggagagacatttgatcccttgtttgcttaataaattataaaatgatggcttggaaaagcaggctagtctaaccatggtgctattattaggcttgcttgttacacacacaggtctaagcctagtatgtcaataaagcaaatacttactgttttgtttctattaatgattcccaaaccttgttgcaagtttttgcattggcatctttggatttcagtcttgatgtttgttctatcagacttaaccttttatttcctgtccttccttgaaattgctgattgttctgctccctctacagatatttatatcaattcctacagctttcccctgccatccctgaactctttctagcccttttagattttggcactgtgaaacccctgctggaaacctgagtgaccctccctccccaccaagagtccacagacctttcatctttcacgaacttgatcctgttagcaggtggtaataccatgggtgctgtgacactaacagtcattgagaggtgggaggaagtcccttttccttggactggtatcttttcaactattgttttatcctgtctttgggggcaatgtgtcaaaagtcccctcaggaattttcagaggaaagaacattttatgaggctttctctaaagtttcctttgtataggagtatgctcacttaaatttacagaaagaggtgagctgtgttaaacctcagagtttaaaagctactgataaactgaagaaagtgtctatattggaactagggtcatttgaaagcttcagtctcggaacatgacctttagtctgtggactccatttaaaaataggtatgaataagatgactaagaatgtaatggggaagaactgccctgcctgcccatctcagagccataaggtcatctttgctagagctatttttacctatgtatttatcgttcttgatcataagccgcttatttatatcatgtatctctaaggacctaaaagcactttatgtagtttttaattaatcttaagatctggttacggtaactaaaaaagcctgtctgccaaatccagtggaaacaagtgcatagatgtgaattggtttttaggggccccacttcccaattcattaggtatgactgtggaaatacagacaaggatcttagttgatattttgggcttggggcagtgagggcttaggacaccccaagtggtttgggaaaggaggaggggagtggtgggtttatagggggaggaggaggcaggtggtctaagtgctgactggctacgtagttcgggcaaatcctccaaaagggaaagggaggatttgcttagaaggatggcgctcccagtgactactttttgacttctgtttgtcttacgcttctctcagggaaaaacatgcagtcctctagtgtttcatgtacattctgtggggggtgaacaccttggttctggttaaacagctgtacttttgatagctgtgccaggaagggttaggaccaactacaaattaatgttggttgtcaaatgtagtgtgtttccctaactttctgtttttcctgagaaaaaaaaataaatcttttattcaaatacagggaaaaaaaaaaaaaaaaaa

SEQ ID NO: 2 - protein, 350 aa, Homo sapiens [sequence omitted]

SEQ ID NO: 3 - DNA, 1048 bp, Homo sapiens
aaggcgggggcgggggcggggcggcggccgtgggtccctgccggccggcggcgggcgcagacagcggcgggcgcaggacgtgcactatggctcggggctcgctgcgccggttgctgcggctcctcgtgctggggctctggctggcgttgctgcgctccgtggccggggagcaagcgccaggcaccgccccctgctcccgcggcagctcctggagcgcggacctggacaagtgcatggactgcgcgtcttgcagggcgcgaccgcacagcgacttctgcctgggctgcgctgcagcacctcctgcccccttccggctgctttggcccatccttgggggcgctctgagcctgaccttcgtgctggggctgctttctggctttttggtctggagacgatgccgcaggagagagaagttcaccacccccatagaggagaccggcggagagggctgcccagctgtggcgctgatccagtgacaatgtgccccctgccagccggggctcgcccactcatcattcattcatccattctagagccagtctctgcctcccagacgcggcgggagccaagctcctccaaccacaaggggggtggggggcggtgaatcacctctgaggcctgggcccagggttcaggggaaccttccaaggtgtctggttgccctgcctctggctccagaacagaaagggagcctcacgctggctcacacaaaacagctgacactgactaaggaactgcagcatttgcacaggggaggggggtgccctccttcctagaggccctgggggccaggctgacttggggggcagacttgacactaggccccactcactcagatgtcctgaaattccaccacgggggtcaccctggggggttagggacctatttttaacactagggggctggcccactaggagggctggccctaagatacagacccccccaactccccaaagcggggaggagatatttattttggggagagtttggaggggagggagaatttattaataaaagaatctttaactttaaaaaaaaaaaaaaaaaa

SEQ ID NO: 4 - protein, 129 aa, Homo sapiens
MARGSLRRLLRLLVLGLWLALLRSVAGEQAPGTAPCSRGSSWSADLDKCMDCASCRARPHSDFCLGCAAAPPAPFRLLWPILGGALSLTFVLGLLSGFLVWRRCRRREKFTTPIEETGGEGCPAVALIQ

SEQ ID NO: 5 - DNA, 24 bp, Artificial Sequence
  Synthetic Sequence
ggactggctagttaaacaaagagg

SEQ ID NO: 6 - DNA, 25 bp, Artificial Sequence
  Synthetic Sequence
cttattagatatgccaaaccagctc

SEQ ID NO: 7 - DNA, 18 bp, Artificial Sequence
  Synthetic Sequence
gctgagttaccggcatcc

SEQ ID NO: 8 - DNA, 24 bp, Artificial Sequence
  Synthetic Sequence
ttctgaagggagagaaagagattc

SEQ ID NO: 9 - DNA, 19 bp, Artificial Sequence
  Synthetic Sequence
ccactaacttctgattcgc

SEQ ID NO: 10 - DNA, 20 bp, Artificial Sequence
  Synthetic Sequence
gcgtgctagctggatgtctt

SEQ ID NO: 11 - DNA, 21 bp, Artificial Sequence
  Synthetic Sequence
cccgccgaattcattaattta

SEQ ID NO: 12 - DNA, 21 bp, Artificial Sequence
  Synthetic Sequence
ccctagcaacctagccagaaa

SEQ ID NO: 13 - DNA, 21 bp, Artificial Sequence
  Synthetic Sequence
ctgcttgggataaagatgcaa

(19) United States
(12) Patent Application Publication
(10) Pub. No.: US 2016/0130654 A1
(43) Pub. Date: May 12, 2016

(54) SYSTEMS AND METHODS FOR DIAGNOSING AND TREATING CANCER

(71) Applicant: THE TRANSLATIONAL GENOMICS RESEARCH INSTITUTE, Phoenix, AZ (US)

(72) Inventors: Timothy G. Whitsett, Phoenix, AZ (US); Nhan L. Tran, Phoenix, AZ (US)

(73) Assignee: THE TRANSLATIONAL GENOMICS RESEARCH INSTITUTE, Phoenix, AZ (US)

(21) Appl. No.: 14/561,304

(22) Filed: Dec. 5, 2014

Related U.S. Application Data
(60) Provisional application No. 61/912,065, filed on Dec. 5, 2013.

(51) Int. Cl. C12Q 1/68; G01N 33/574; A61K 33/24; A61K 31/4709; A61K 31/7088; C12N 15/113

(52) U.S. Cl. CPC C12Q 1/6886; A61K 31/7088; C12N 15/1135; A61K 33/24; A61K 31/4709; G01N 33/57423; C12N 2310/14; C12N 2320/31; C12Q 2600/158; C12Q 2600/112; G01N 2333/4703; G01N 2333/70578

(57) ABSTRACT

Embodiments of the invention provide a method detecting and treating cancer, such as lung cancer. In some aspects, the method may include detecting cancer in a subject, which may comprise assessing the expression of a marker in a sample from the subject. For example, the marker may comprise Mcl-1. In some embodiments, if the subject is diagnosed as having cancer, the method may further provide administering a therapeutically effective amount of a substance that reduces the expression level of Mcl-1 to the subject and then administering a treatment modality that is known to treat the cancer.

### CROSS-REFERENCE TO RELATED APPLICATIONS

The present application claims priority to U.S. Patent Application No. 61/912,065 filed Dec. 5, 2013, which is hereby incorporated by reference in its entirety.

### STATEMENT REGARDING FEDERALLY SPONSORED RESEARCH

This invention was made with government support under CA130940 awarded by the National Institutes of Health. The government has certain rights in the invention.

### INCORPORATION-BY-REFERENCE OF MATERIAL ELECTRONICALLY FILED

Incorporated by reference in its entirety herein is a computer-readable nucleotide/amino acid sequence listing submitted concurrently herewith and identified as follows: One 13 kilobyte ASCII (text) file named “Mcl_Fn14_ST25” created Dec. 2, 2014.

### FIELD OF THE INVENTION

The present invention is generally related to systems and methods for diagnosing and treating one or more forms of cancer, and particularly related to systems and methods for diagnosing and treating non-small cell lung cancer.

### BACKGROUND OF THE INVENTION

Lung cancer is the leading cause of cancer-related mortality in the USA and throughout the world, with a five-year survival rate for advanced, non-small cell lung cancer (NSCLC), the most common class of lung cancer, below 10%, in part due to intrinsic and acquired resistance to standard therapeutics (Heist R S, Engelman J A. SnapShot: non-small cell lung cancer. Cancer Cell. 2012; 21:448 e2). While targeted therapies have shown promise in small subsets of patients, the majority of lung cancer patients rely on platinum-derived chemotherapeutics and radiation therapy in the absence of more effective targeted therapeutics. Acquired resistance to these treatments remains a significant barrier to reducing mortality in NSCLC patients (Chang A. Chemotherapy, chemoresistance and the changing treatment landscape for NSCLC. Lung Cancer. 2011; 71:3-10; Hildebrandt M A, Gu J, Wu X. Pharmacogenomics of platinum-based chemotherapy in NSCLC. Expert Opin Drug Metab Toxicol. 2009; 5:745-55). A deeper understanding of the molecular events leading to therapeutic resistance could aid in identifying novel therapeutic targets to improve patient prognosis in advanced NSCLC.

The tumor necrosis factor-like weak inducer of apoptosis (TWEAK)-fibroblast growth factor-inducible 14 (Fn14; TNFRSF12a) signaling axis has been implicated in a number of solid tumor types and can affect tumor cell proliferation, apoptosis, cell invasion, and cell survival (Winkles J A. The TWEAK-Fn14 cytokine-receptor axis: discovery, biology and therapeutic targeting. Nat Rev Drug Discov. 2008; 7:411-25). In NSCLC, Fn14 is over-expressed in primary tumors, correlated with activated EGFR, and promoted tumor cell migration and invasion (Whitsett T G, Cheng E, Inge L, Asrani K, Jameson N M, Hostetter G, et al. Elevated expression of Fn14 in non-small cell lung cancer correlates with activated EGFR and promotes tumor cell migration and invasion. Am J Pathol. 2012; 181:111-20). In glioblastoma (GB), TWEAK exposure resulted in enhanced tumor cell invasion through Rac1 and NF-κB activation (Tran N L, McDonough W S, Savitch B A, Fortin S P, Winkles J A, Symons M, et al. Increased fibroblast growth factor-inducible 14 expression levels promote glioma cell invasion via Rac1 and nuclear factor-kappaB and correlate with poor patient outcome. Cancer Res. 2006; 66:9535-42). In addition, TWEAK-Fn14 signaling promoted GB cell survival, primarily through Akt2 phosphorylation, NF-κB activation, and up-regulation of Bcl-2 family members such as Bcl-xL and Bcl-w (Fortin S P, Ennis M J, Savitch B A, Carpentieri D, McDonough W S, Winkles J A, et al. Tumor necrosis factor-like weak inducer of apoptosis stimulation of glioma cell survival is dependent on Akt2 function. Mol Cancer Res. 2009; 7:1871-81; Tran N L, McDonough W S, Savitch B A, Sawyer T F, Winkles J A, Berens M E. The tumor necrosis factor-like weak inducer of apoptosis (TWEAK)-fibroblast growth factor-inducible 14 (Fn14) signaling system regulates glioma cell survival via NFkappaB pathway activation and BCL-XL/BCL-W expression. J Biol Chem. 2005; 280:3483-92). The role and mechanism(s) of TWEAK-mediated tumor cell survival in NSCLC has not been described.

Pro-survival members of the Bcl-2 family, including Bcl-2, Bcl-xL, Bcl-w, and Mcl-1, are elevated in numerous cancer types and contribute to cancer cell survival and resistance to therapy, largely through direct inhibition of pro-apoptotic Bcl-2 family members (Kelly P N, Strasser A. The role of Bcl-2 and its pro-survival relatives in tumourigenesis and cancer therapy. Cell Death Differ. 2011; 18:1414-24). Mcl-1 is a mitochondria-associated pro-survival Bcl-2 family member first characterized as a potent, short-term promoter of cell survival during myeloid cell differentiation (Kozopas K M, Yang T, Buchan H L, Zhou P, Craig R W. MCL1, a gene expressed in programmed myeloid cell differentiation, has sequence similarity to BCL2. Proc Natl Acad Sci USA. 1993; 90:3516-20). Mcl-1 is often found to be over-expressed in NSCLC lines compared to normal lung and correlated with poor patient prognosis (Borner M M, Brousset P, Pfanner-Meyer B, Bacchi M, Vonlanthen S, Hotz M A, et al. Expression of apoptosis regulatory proteins of the Bcl-2 family and p53 in primary resected non-small-cell lung cancer. Br J Cancer. 1999; 79:952-8; Luo L, Zhang T, Liu H, Lv T, Yuan D, Yao Y, et al. MiR-101 and Mcl-1 in non-small-cell lung cancer: expression profile and clinical significance. Med Oncol. 2012; 29:1681-6). Mcl-1 binds pro-apoptotic Bcl-2 family members Noxa, Bak, and Bax, thus maintaining their inactive monomeric state and limiting apoptotic signaling, especially in NSCLC lines with high expression of Mcl-1 (Zhang H, Guttikonda S, Roberts L, Uziel T, Semizarov D, Elmore S W, et al. Mcl-1 is critical for survival in a subgroup of non-small-cell lung cancer cell lines. Oncogene. 2011; 30:1963-8). Further, EGF/ERK signaling induced Mcl-1 and protected NSCLC cells against TKI and chemotherapeutic-induced cell death, with the depletion of Mcl-1 conferring increased sensitization to radiation and chemotherapeutic insult (Song L, Coppola D, Livingston S, Cress D, Haura E B. Mcl-1 regulates survival and sensitivity to diverse apoptotic stimuli in human non-small cell lung cancer cells. Cancer Biol Ther. 2005; 4:267-76). Mcl-1 has been additionally implicated in PI3K/Akt pro-survival signaling in NSCLC; Akt2 knockdown induces Mcl-1 cleavage and mitochondrial-driven cell death (Lee M W, Kim D S, Lee J H, Lee B S, Lee S H, Jung H L, et al. Roles of AKT1 and AKT2 in non-small cell lung cancer cell survival, growth, and migration. Cancer Sci. 2011; 102:1822-8), and PI3K inhibition leads to decreased Mcl-1 in EGFR mutant lines (Faber A C, Li D, Song Y, Liang M C, Yeap B Y, Bronson R T, et al. Differential induction of apoptosis in HER2 and EGFR addicted cancers following PI3K inhibition. Proc Natl Acad Sci USA. 2009; 106:19503-8). In an in vivo model of NSCLC driven by c-Myc over-expression and mutant KRAS, Mcl-1 up-regulation was found to be necessary for evasion of apoptosis (Allen T D, Zhu C Q, Jones K D, Yanagawa N, Tsao M S, Bishop J M. Interaction between MYC and MCL1 in the genesis and outcome of non-small-cell lung cancer. Cancer Res. 2011; 71:2212-21). Thus, Mcl-1 may play a role in NSCLC cell survival through antagonizing apoptotic signaling, and could be a novel therapeutic target towards improved efficacy of cytotoxic therapies.

In view of the aforementioned, there is a need to further understand the TWEAK-Fn14 pro-survival signaling axis in NSCLC and its potential dependence on Mcl-1. Moreover, there is also a need to assess the role of TWEAK in conferring protection of NSCLC cells against forms of treatment, such as radiation and chemotherapeutic modalities. In addition, there is also a need to assess the impact of augmenting levels of Mcl-1 on cell survival.

### SUMMARY OF THE INVENTION

Some embodiments of the invention provide a method of diagnosing cancer. For example, the method may include receiving a sample from a subject suspected of having cancer and detecting a level of expression of Mcl-1 and Fn14 in the sample from the subject. In some aspects, the levels of expression of Mcl-1 and Fn14 may be expressed relative to Mcl-1 and Fn14 expression levels in a control sample. In some aspects, the method may further comprise diagnosing the subject as having cancer when the levels of expression of Mcl-1 and Fn14 are both elevated compared to the control sample. In some embodiments, the cancer may be a form of lung cancer, such as non-small cell lung cancer.

After diagnosis, some embodiments of the invention further provide administering a therapeutically effective amount of a first pharmaceutical composition that inhibits Mcl-1 to the subject and then administering a treatment modality that is known to treat the cancer. For example, the treatment modality may comprise radiation or a therapeutically effective amount of a second pharmaceutical composition that may comprise a chemotherapeutic agent, such a cisplatin.

Some embodiments of the invention provide a method of predicting a cancer stage in a subject that has been diagnosed with cancer. For example the method may include receiving a sample from a subject that has been diagnosed with cancer, detecting a level of expression of Mcl-1 in the sample from the subject and also detecting a level of expression of Fn14 in the sample from the subject. In some aspects, the levels of expression of Mcl-1 and Fn14 are determined relative to the expression of Mcl-1 and Fn14 in a control sample. In some embodiments, the level of expression of Mcl-1 and Fn14 are correlated with the cancer stage. For example, in some aspects, greater expression levels of Mcl-1 and Fn14 can be correlated with a more advanced tumor stage. In some embodiments, the cancer may be a form of lung cancer, such as non-small cell lung cancer. In some aspects, detection of Mcl-1 and Fn14 expression levels may be accomplished using at least one of an antibody and an oligonucleotide.

Other embodiments of the invention provide a method of diagnosing and treating cancer, which can include diagnosing a subject as having cancer and then treating the cancer in subjects diagnosed as having the cancer. In some aspects, detecting the cancer includes receiving a sample from the subject suspected of having cancer, adding a first reagent capable of binding to Fn14 to a mixture comprising the sample, and subjecting the mixture to conditions that allow detection of the binding. In some embodiments, the cancer may be a form of lung cancer, such as non-small cell lung cancer.

Thereafter, if the subject is diagnosed as having cancer, the method of treatment includes administering a therapeutically effective amount of a first pharmaceutical composition that inhibits Mcl-1 to the subject, and administering a treatment modality that is known to treat the cancer. For example, the treatment modality may comprise radiation or a therapeutically effective amount of a second pharmaceutical composition that may comprise a chemotherapeutic agent, such a cisplatin. In some aspects, the substance that reduces the expression level of Mcl-1 comprises small RNAs targeting SEQ ID NO: 1 and/or at least one pharmacological inhibitor. In addition, in some embodiments, the method may further comprise adding a second reagent capable of binding to Mcl-1 to a mixture comprising the sample and subjecting the mixture to conditions that allow detection of the binding the second reagent.

Additional objectives, advantages and novel features will be set forth in the description which follows or will become apparent to those skilled in the art upon examination of the drawings and detailed description which follows.

### BRIEF DESCRIPTION OF THE DRAWINGS

FIGS. 1A and 1B illustrate that Mcl-1 expression in human NSCLC specimens correlates with Fn14 expression. (FIG. 1A) Mcl-1 and Fn14 staining on representative samples from the same patient with lung adenocarcinoma (5× objective, Aperio GL Scanner). Tumor-cell specific Fn14 and Mcl-1 staining in each of the tumor punches was scored by a board-certified pathologist; a score of zero indicates staining level equal to adjacent non-tumor cells. A non-zero score indicates increased staining (1=minimum, 2=moderate, 3=strong positive). (FIG. 1B) A total of 290 samples were scored for Mcl-1 and Fn14 expression and the correlation between the two stains was analyzed using Kendall's tau test.

FIG. 2 illustrates that Mcl-1 and Fn14 gene expressions correlate in squamous cell lung carcinoma. The mRNA expressions of Mcl-1 and Fn14 were examined in squamous cell lung carcinoma (n=44) using publically available gene expression data (www.genesapiens.org). Statistical correlations are performed on the website.

FIGS. 3A and 3B illustrate that Mcl-1 gene expression correlates with higher stage and worse prognosis in NSCLC. The mRNA expression of Mcl-1 was evaluated in the Bild Lung data set (www.oncomine.org) for correlations with (FIG. 3A) advancing clinical stage of lung adenocarcinoma and (FIG. 3B) patient mortality after one year. Data are presented as box-and-whisker plots. The box represents the interquartile range (25-75th percentile) and the line within this box is the median value. Bottom and top bars of the whisker indicate the 10th and 90th percentiles, respectively. Maximum/minimum values are indicated (•). Statistical significance (**) as defined by a p value <0.05 is determined in Oncomine.

FIGS. 4A-4C illustrate that TWEAK induces Mcl-1 in NSCLC cell lines in an NF-κB-dependent manner. Total cell lysates were prepared from serum-reduced (FIG. 4A) H1975 and (FIG. 4B) H2073 cell lines treated with TWEAK for the indicated times and immunoblotted with the indicated antibodies: Mcl-1, Bcl-xL, and phosphorylated-p65 (Ser536). Tubulin was used as a loading control. (FIG. 4C) Serum-reduced H2073 cells transfected±IkBα mutant were treated with TWEAK for 24 hours. Cells were harvested, total cell lysates were prepared and immunoblotted with the indicated antibodies to both Mcl-1 and phospho-p65. All blots were run in duplicate and tubulin was used as a loading control.

FIGS. 5A and 5B illustrate that TWEAK exposure enhances Mcl-1 and Bcl-xL mRNA expression. Total RNA was collected from (FIG. 5A) H1975 and (FIG. 5B) H2073 cells treated with 100 ng/mL TWEAK for the indicated time points. mRNA expression of Mcl-1 and Bcl-xL were determined by qPCR with histone H3.3 mRNA levels used as endogenous control. Bars represent the average±standard error of triplicate qPCR reactions. * represents a p value <0.05 compared to no treatment (of the respective gene) by Student's t test. All assays were run in duplicate.

FIGS. 6A-6F illustrate that TWEAK-induced NSCLC cell survival is dependent on Mcl-1 expression. H1975 (FIG. 6A) and H2073 (FIG. 6B) cells were transfected with luciferase (siCont) or siRNAs targeting Mcl-1. Total lysates were collected 72 hours post-transfection and immunoblotted for Mcl-1 and alpha-tubulin. H1975 (FIGS. 6C and 6E) and H2073 (FIGS. 6D and 6F) cells transfected with control or siRNA constructs targeting Mcl-1 were exposed to 1 μM cisplatin for 24 hours (FIGS. 6C and 6D) or 2Gy ionizing radiation (FIGS. 6E and 6F)±pre-incubation with TWEAK (100 ng/mL). Cells were sparsely seeded into 6-well dishes and allowed to grow for 7 days prior to staining with crystal violet and colony counting. A colony was defined as containing at least 50 cells. Bars represent average of three independent wells±standard error with the non-treated (first bar) set to 1. * represents a p value <0.05 by ANOVA with Bonferroni posttest.

FIGS. 7A-7F illustrate that Bcl-xL depletion sensitizes lung cancer cells to therapeutic insult but does not fully rescue TWEAK-induced cell survival. H1975 (FIG. 7A) and H2073 (FIG. 7B) cells were transfected with luciferase (siCont) or siRNA targeting Bcl-xL. Total lysates were collected 72 hours post-transfection and immunoblotted for Bcl-xL and alpha-tubulin. H1975 (FIGS. 7C and 7E) and H2073 (FIGS. 7D and 7F) cells transfected with control or siRNA construct targeting Bcl-xL were exposed to 1 μM cisplatin for 24 hours (FIGS. 7C and 7D) or 2Gy ionizing radiation (FIGS. 7E and 7F)±pre-incubation with TWEAK (100 ng/mL). Cells were sparsely seeded into 6-well dishes and allowed to grow for 7 days prior to staining with crystal violet and colony counting. A colony was defined as containing at least 50 cells. Bars represent average of three independent wells±standard error with the non-treated (first bar) set to 1. * represents a p value <0.05 by ANOVA with Bonferroni posttest.

FIGS. 8A and 8B illustrate that depletion of Mcl-1 abrogates TWEAK-induced protection from cell death induced by DNA damage. H1975 cells were transfected with either siRNA targeting luciferase (control) or Mcl-1. Cells were exposed to 5 uM cisplatin (FIG. 8A) or 8Gy radiation (FIG. 8B) for 0, 4 or 24 hours±pre-incubation with TWEAK (100 ng/mL). Total cell lysates were prepared and immunoblotted for cleaved-PARP (cPARP) and GAPDH as a loading control. All blots were run in duplicate.

FIGS. 9A-9C illustrate that pharmacologic inhibition of Mcl-1 inhibits NSCLC cell growth. (FIG. 9A) H1975 cells were grown in the presence or absence of TWEAK (100 ng/mL) and EU-5148 (10-M). Cells were lysed and immunoprecipitated with anti-Bak antibodies. Protein expression Mcl-1, Bcl-xL and Bak after immunoprecipitation were resolved by immunoblot analysis. (FIG. 9B) Cell viability of DHL10, Bcl-2 1863 and Mcl-1 1780 cells was assessed by PrestoBlue assay. Cells were exposed to the indicated concentrations of EU-5148 in DMSO for 48 hours. Cell killing curves and EC-50 values were generated from triplicate runs in GraphPad Prism 5. (FIG. 9C) A panel of NSCLC cell lines was exposed to vehicle or 10 μM EU-5148 for 48 hours. Cell growth was assessed by Cell-Titer Glo assay. Bars represent the average of two wells with the untreated set to 100%.

FIGS. 10A and 10B illustrate that EU-5148 inhibits Mcl-1-Bim protein interaction. The protein interactions of Mcl-1 and Bim (FIG. 10A) and Bcl-xL and Bim (FIG. 10B) were assayed by a competitive displacement ELISA assay. Mcl-1-GST or Bcl-xL-GST were incubated with biotinyated BIM with and without the indicated concentrations of EU-5148 for two hours. Absorbance was measured at 450 nm and binding curves were plotted using GraphPad Prism 5.

FIGS. 11A and 11B illustrate that pharmacologic inhibition of Mcl-1 abrogates TWEAK-mediated cell survival. H1975 (FIGS. 11A and 11B) cells were pre-incubated with TWEAK (100 ng/mL), (FIG. 11A) EU-5148 (10 μM), (FIG. 11B) ABT-737 (10 ▪M) or both drug and TWEAK prior to exposure to 2Gy ionizing radiation. Cells were sparsely seeded (125 cells) into 6-well dishes and allowed to grow for 7 days prior to staining with crystal violet and colony counting. A colony was defined as containing at least 50 cells. Bars represent average of three independent wells±standard error with the non-treated (first bar) set to 1. * represents a p value <0.05 by ANOVA with Bonferroni posttest.

FIGS. 12A-12C illustrate that pharmacologic inhibition of Mcl-1 abrogates TWEAK-mediated cell survival. H1975 (FIG. 12A) and H2073 (FIG. 12B-C) cells were pre-incubated with TWEAK (100 ng/mL), EU-5148 (10 μM) or both prior to exposure to 1 μM cisplatin (FIG. 12A-B) or 2Gy ionizing radiation (FIG. 12C). Cells were sparsely seeded (125 cells) into 6-well dishes and allowed to grow for 7 days prior to staining with crystal violet and colony counting. A colony was defined as containing at least 50 cells. Bars represent average of three independent wells±standard error with the non-treated (first bar) set to 1. * represents a p value <0.05 by one-way ANOVA with multiple comparison testing.

The headings used in the figures should not be interpreted to limit the scope of the claims.

### DETAILED DESCRIPTION

Some embodiments of the invention provide methods of detecting, diagnosing, and/or treating a disease, such as cancer. Moreover, some embodiments of the invention provide methods of assessing the progress of the disease and/or predicting patient outcome/decline. For example, some embodiments of the invention include methods of diagnosing cancer and/or treating the cancer. In some aspects, the cancer may comprise lung cancer, which may further include non-small cell lung cancer. Some embodiments of the invention may also include determining the relative stage of a tumor that is associated with the cancer. Some embodiments comprise the use of detecting, quantifying, and/or augmenting the presence of one or more markers. In some embodiments of the invention, the marker may comprise Mcl-1 and/or Fn14. In some aspects, the marker may comprise Mcl-1 nucleic acids (SEQ ID NO: 1) or protein (SEQ ID NO: 2) and/or Fn14 nucleic acids (SEQ ID NO: 3) or protein (SEQ ID NO: 4). In particular, some embodiments include augmenting (e.g., increasing or decreasing) a level of expression of the one or more markers and then providing a therapeutic modality to a patient to treat the cancer.

Generally, some embodiments of the present invention can be used to identify, quantify, detect, assess, isolate, and/or augment expression levels of one or more markers. A marker may be any molecular structure produced by a cell, expressed inside the cell, accessible on the cell surface, or secreted by the cell. A marker may be any protein, carbohydrate, fatty acid, nucleic acid, catalytic site, or any combination of these such as an enzyme, glycoprotein, cell membrane, virus, a particular cell, or other uni- or multimolecular structure. A marker may be represented by a sequence of a nucleic acid or any other molecules derived from the nucleic acid. Examples of such nucleic acids include miRNA, tRNA, siRNA, mRNA, cDNA, genomic DNA sequences, or complementary sequences thereof. Alternatively, a marker may be represented by a protein sequence. The concept of a marker is not limited to the exact nucleic acid sequence or protein sequence or products thereof, rather it encompasses all molecules that may be detected by a method of assessing the marker. Without being limited by the theory, the detection of the marker may encompass the detection and/or determination of a change in copy number (e.g., copy number of a gene or other forms of nucleic acid) or in the detection of one or more translocations.

Therefore, examples of molecules encompassed by a marker represented by a particular sequence further include alleles of the gene used as a marker. An allele includes any form of a particular nucleic acid that may be recognized as a form of the particular nucleic acid on account of its location, sequence, or any other characteristic that may identify it as being a form of the particular gene. Alleles include but need not be limited to forms of a gene that include point mutations, silent mutations, deletions, frame shift mutations, single nucleotide polymorphisms (SNPs), inversions, translocations, heterochromatic insertions, and differentially methylated sequences relative to a reference gene, whether alone or in combination.

An allele of a gene may or may not produce a functional protein; may produce a protein with altered function, localization, stability, dimerization, or protein-protein interaction; may have overexpression, under-expression or no expression; may have altered temporal or spatial expression specificity; or may have altered copy number (e.g., greater or less numbers of copies of the allele). An allele may also be called a mutation or a mutant. An allele may be compared to another allele that may be termed a wild type form of an allele. In some cases, the wild type allele is more common than the mutant.

Some embodiments of the invention may comprise the use of one or more methods of amplifying a nucleic acid-based starting material (i.e., a template). Nucleic acids may be selectively and specifically amplified from a template nucleic acid contained in a sample. In some nucleic acid amplification methods, the copies are generated exponentially. Examples of nucleic acid amplification methods known in the art include: polymerase chain reaction (PCR), ligase chain reaction (LCR), self-sustained sequence replication (3SR), nucleic acid sequence based amplification (NASBA), strand displacement amplification (SDA), amplification with Qβ replicase, whole genome amplification with enzymes such as φ29, whole genome PCR, in vitro transcription with T7 RNA polymerase or any other RNA polymerase, or any other method by which copies of a desired sequence are generated.

In addition to genomic DNA, any oligonucleotide or polynucleotide sequence can be amplified with an appropriate set of primer molecules. In particular, the amplified segments created by the PCR process itself are, themselves, efficient templates for subsequent PCR amplifications. For example, as described in greater detail herein, in some aspects of the invention, a first reagent can be used to detect Mcl-1 and a second reagent can be used to detect Fn14. In some embodiments, the first and/or the second reagents may comprise one or more oligonucleotides (e.g., primers) that can specifically bind to DNA, RNA, and/or cDNA to detect the presence and/or expression of nucleic acids that correspond to Mcl1 (SEQ ID NO: 1) and/or Fn14 (SEQ ID NO: 3).

PCR generally involves the mixing of a nucleic acid sample, two or more primers that are designed to recognize the template DNA, a DNA polymerase, which may be a thermostable DNA polymerase such as Taq or Pfu, and deoxyribose nucleoside triphosphates (dNTP's). Reverse transcription PCR, quantitative reverse transcription PCR, and quantitative real time reverse transcription PCR are other specific examples of PCR. In general, the reaction mixture is subjected to temperature cycles comprising a denaturation stage (typically 80-100° C.), an annealing stage with a temperature that is selected based on the melting temperature (Tm) of the primers and the degeneracy of the primers, and an extension stage (for example 40-75° C.). In real-time PCR analysis, additional reagents, methods, optical detection systems, and devices known in the art are used that allow a measurement of the magnitude of fluorescence in proportion to concentration of amplified DNA. In such analyses, incorporation of fluorescent dye into the amplified strands may be detected or measured.

Alternatively, labeled probes that bind to a specific sequence during the annealing phase of the PCR may be used with primers. Labeled probes release their fluorescent tags during the extension phase so that the fluorescence level may be detected or measured. Generally, probes are complementary to a sequence within the target sequence downstream from either the upstream or downstream primer. Probes may include one or more label. A label may be any substance capable of aiding a machine, detector, sensor, device, or enhanced or unenhanced human eye from differentiating a labeled composition from an unlabeled composition. Examples of labels include but are not limited to: a radioactive isotope or chelate thereof, dye (fluorescent or nonfluorescent,) stain, enzyme, or nonradioactive metal. Specific examples include, but are not limited to: fluorescein, biotin, digoxigenin, alkaline phosphatese, biotin, streptavidin, ³H, ¹⁴C, ³²P, ³⁵S, or any other compound capable of emitting radiation, rhodamine, 4-(4′-dimethylamino-phenylazo) benzoic acid (“Dabcyl”); 4-(4′-dimethylamino-phenylazo)sulfonic acid (sulfonyl chloride) (“Dabsyl”); 5-((2-aminoethyl)-amino)-naphtalene-1-sulfonic acid (“EDANS”); Psoralene derivatives; haptens, cyanines, acridines, fluorescent rhodol derivatives, cholesterol derivatives; ethylenediaminetetraaceticacid (“EDTA”) and derivatives thereof or any other compound that may be differentially detected. The label may also include one or more fluorescent dyes optimized for use in genotyping. Examples of dyes facilitating the reading of the target amplification include, but are not limited to: CAL-Fluor Red 610, CAL-Fluor Orange 560, dR110, 5-FAM, 6FAM, dR6G, JOE, HEX, VIC, TET, dTAMRA, TAMRA, NED, dROX, PET, BHQ+, Gold540, and LIZ.PCR facilitating the reading of the target amplification.

Either primers or primers along with probes allow a quantification of the amount of specific template DNA present in the initial sample. In addition, RNA may be detected by PCR analysis by first creating a DNA template from RNA through a reverse transcriptase enzyme. The marker expression may be detected by quantitative PCR analysis facilitating genotyping analysis of the samples.

An illustrative example, using dual-labeled oligonucleotide probes in PCR reactions is disclosed in U.S. Pat. No. 5,716,784 to DiCesare. In one example of the PCR step of the multiplex Real Time-PCR/PCR reaction of the present invention, the dual-labeled fluorescent oligonucleotide probe binds to the target nucleic acid between the flanking oligonucleotide primers during the annealing step of the PCR reaction. The 5′ end of the oligonucleotide probe contains the energy transfer donor fluorophore (reporter fluor) and the 3′ end contains the energy transfer acceptor fluorophore (quenching fluor). In the intact oligonucleotide probe, the 3′ quenching fluor quenches the fluorescence of the 5′ reporter fluor. However, when the oligonucleotide probe is bound to the target nucleic acid, the 5′ to 3′ exonuclease activity of the DNA polymerase, e.g., Taq DNA polymerase, will effectively digest the bound labeled oligonucleotide probe during the amplification step. Digestion of the oligonucleotide probe separates the 5′ reporter fluor from the blocking effect of the 3′ quenching fluor. The appearance of fluorescence by the reporter fluor is detected and monitored during the reaction, and the amount of detected fluorescence is proportional to the amount of fluorescent product released. Examples of apparatus suitable for detection include, e.g. Applied Biosystems™ 7900HT real-time PCR platform and Roche's 480 LightCycler, the ABI Prism 7700 sequence detector using 96-well reaction plates or GENEAMP PC System 9600 or 9700 in 9600 emulation mode followed by analysis in the ABA Prism Sequence Detector or TAQMAN LS-50B PCR Detection System. The labeled probe facilitated multiplex Real Time-PCR/PCR can also be performed in other real-time PCR systems with multiplexing capabilities.

“Amplification” is a special case of nucleic acid replication involving template specificity. Amplification may be a template-specific replication or a non-template-specific replication (i.e., replication may be specific template-dependent or not). Template specificity is here distinguished from fidelity of replication (synthesis of the proper polynucleotide sequence) and nucleotide (ribo- or deoxyribo-) specificity. Template specificity is frequently described in terms of “target” specificity. Target sequences are “targets” in the sense that they are sought to be sorted out from other nucleic acid. Amplification techniques have been designed primarily for this sorting out.

The term “template” refers to nucleic acid originating from a sample that is analyzed for the presence of a marker of interest. In contrast, “background template” or “control” is used in reference to nucleic acid other than sample template that may or may not be present in a sample. Background template is most often inadvertent. It may be the result of carryover, or it may be due to the presence of nucleic acid contaminants sought to be purified out of the sample. For example, nucleic acids from organisms other than those to be detected may be present as background in a test sample.

In addition to primers and probes, template specificity is also achieved in some amplification techniques by the choice of enzyme. Amplification enzymes are enzymes that, under the conditions in which they are used, will process only specific sequences of nucleic acid in a heterogeneous mixture of nucleic acid. Other nucleic acid sequences will not be replicated by this amplification enzyme. Similarly, in the case of T7 RNA polymerase, this amplification enzyme has a stringent specificity for its own promoters (Chamberlin et al. (1970) Nature (228):227). In the case of T4 DNA ligase, the enzyme will not ligate the two oligonucleotides or polynucleotides, where there is a mismatch between the oligonucleotide or polynucleotide substrate and the template at the ligation junction (Wu and Wallace (1989) Genomics (4):560). Finally, Taq and Pfu polymerases, by virtue of their ability to function at high temperature, are found to display high specificity for the sequences bounded and thus defined by the primers; the high temperature results in thermodynamic conditions that favor primer hybridization with the target sequences and not hybridization with non-target sequences (H. A. Erlich (ed.) (1989) PCR Technology, Stockton Press).

The term “amplifiable nucleic acid” refers to nucleic acids that may be amplified by any amplification method. It is contemplated that “amplifiable nucleic acid” will usually comprise “sample template.” The terms “PCR product,” “PCR fragment,” and “amplification product” refer to the resultant mixture of compounds after two or more cycles of the PCR steps of denaturation, annealing and extension. These terms encompass the case where there has been amplification of one or more segments of one or more target sequences.

In some forms of PCR assays, quantification of a target in an unknown sample is often required. Such quantification is often in reference to the quantity of a control sample. The control sample DNA may be co-amplified in the same tube in a multiplex assay or may be amplified in a separate tube. Generally, the control sample contains DNA at a known concentration. The control sample DNA may be a plasmid construct comprising only one copy of the amplification region to be used as quantification reference. To calculate the quantity of a target in an unknown sample, various mathematical models are established. Calculations are based on the comparison of the distinct cycle determined by various methods, e.g., crossing points (CP) and cycle threshold values (Ct) at a constant level of fluorescence; or CP acquisition according to established mathematic algorithm.

The algorithm for Ct values in real time-PCR calculates the cycle at which each PCR amplification reaches a significant threshold. The calculated Ct value is proportional to the number of target copies present in the sample, and the Ct value is a precise quantitative measurement of the copies of the target found in any sample. In other words, Ct values represent the presence of respective target that the primer sets are designed to recognize. If the target is missing in a sample, there should be no amplification in the Real Time-PCR reaction.

Alternatively, the Cp value may be utilized. A Cp value represents the cycle at which the increase of fluorescence is highest and where the logarithmic phase of a PCR begins. The LightCycler® 480 Software calculates the second derivatives of entire amplification curves and determines where this value is at its maximum. By using the second-derivative algorithm, data obtained are more reliable and reproducible, even if fluorescence is relatively low.

The various and non-limiting embodiments of the PCR-based method detecting marker expression level as described herein may comprise one or more probes and/or primers. Generally, the probe or primer contains a sequence complementary to a sequence specific to a region of the nucleic acid of the marker gene. A sequence having less than 60% 70%, 80%, 90%, 95%, 99% or 100% identity to the identified gene sequence may also be used for probe or primer design if it is capable of binding to its complementary sequence of the desired target sequence in marker nucleic acid.

An oligonucleotide may be any polynucleotide of at least 2 nucleotides. Oligonucleotides may be less than 10, 15, 20, 30, 40, 50, 75, 100, 200, or 500 nucleotides in length. While oligonucleotides are often linear, they may assume a circular or other two dimensional structure. Oligonucleotides may be chemically synthesized by any of a number of methods including sequential synthesis, solid phase synthesis, or any other synthesis method now known or yet to be disclosed. Alternatively, oligonucleotides may be produced by recombinant DNA based methods. In some aspects of the invention, an oligonucleotide may be 2 to 1000 bases in length. In other aspects, it may be 5 to 500 bases in length, 5 to 100 bases in length, 5 to 50 bases in length, or 10 to 30 bases in length. One skilled in the art would understand the length of oligonucleotide necessary to perform a particular task. Oligonucleotides may be directly labeled, used as primers in PCR or sequencing reactions, or bound directly to a solid substrate as in oligonucleotide arrays. For example, as described in greater detail herein, in some aspects of the invention, a first reagent can be used to detect Mcl-1 and a second reagent can be used to detect Fn14. In some embodiments, the first and/or the second reagents may comprise one or more oligonucleotides (e.g., primers) that can specifically bind to DNA, RNA, and/or cDNA to detect the presence and/or expression of nucleic acids that correspond to Mcl1 (SEQ ID NO: 1) and/or Fn14 (SEQ ID NO: 3).

Some embodiments of the invention may include assessing, determining, quantifying, or altering the expression of a marker. As used herein expression encompasses any and all processes through which material derived from a nucleic acid template may be produced. Expression thus includes RNA transcription, mRNA splicing, protein translation, protein folding, post-translational modification, membrane transport, associations with other molecules, addition of carbohydrate moieties to proteins, phosphorylation, protein complex formation and any other process along a continuum that results in biological material derived from genetic material. Expression also encompasses all processes through which the production of material derived from a nucleic acid template may be actively or passively suppressed. Such processes include all aspects of transcriptional and translational regulation. Examples include heterochromatic silencing, transcription factor inhibition, any form of RNAi silencing, microRNA silencing, small interfering RNA silencing, alternative splicing, protease digestion, posttranslational modification, and alternative protein folding.

Expression may be assessed by any number of methods used to detect material derived from a nucleic acid template used currently in the art and yet to be developed. Examples of such methods include any nucleic acid detection method including the following nonlimiting examples, microarray analysis, RNA in situ hybridization, RNAse protection assay, Northern blot, reverse transcriptase PCR, quantitative PCR, quantitative reverse transcriptase PCR, quantitative real-time reverse transcriptase PCR, reverse transcriptase treatment followed by direct sequencing, or any other method of detecting a specific nucleic acid now known or yet to be disclosed.

Other examples include any process of assessing expression that uses an antibody to detect protein expression of the markers, including the following nonlimiting examples, flow cytometry, immunohistochemistry, ELISA, Western blot, and immunoaffinity chromatography. Antibodies may be monoclonal, polyclonal, or any antibody fragment including an Fab, F(ab)₂, Fv, scFv, phage display antibody, peptibody, multispecific ligand, or any other reagent with specific binding to a marker. Such methods also include direct methods used to assess protein expression including the following nonlimiting examples: HPLC, mass spectrometry, protein microarray analysis, PAGE analysis, isoelectric focusing, 2-D gel electrophoresis, and enzymatic assays. For example, as described in greater detail herein, in some aspects of the invention, a first reagent can be used to detect Mcl-1 and a second reagent can be used to detect Fn14, In some embodiments, the first and/or the second reagents may comprise one or more antibodies that can specifically bind to protein to detect the presence and/or expression of proteins that correspond to Mcl1 (SEQ ID NO: 2) and/or Fn14 (SEQ ID NO: 4). For example, the first and second reagents in the protein context can be assessed using techniques such as immunohistochemistry, western blot analysis, flow cytometry, ELISA, and immunoaffinity chromatography. Samples from which expression may be detected include single cells, whole organs or any fraction of a whole organ, whether in vitro, ex vivo, in vivo, or post-mortem.

Other methods used to assess expression include the use of natural or artificial ligands capable of specifically binding one or more markers, including a protein, carbohydrate, fat, nucleic acid, catalytic site, or any combination of these such as an enzyme, glycoprotein, cell membrane, virus, cell, organ, organelle, or any uni- or multimolecular structure that constitutes a marker that may be specifically bound by a ligand. Such ligands include antibodies, antibody complexes, conjugates, natural ligands, small molecules, nanoparticles, or any other molecular entity capable of specific binding to a marker. Ligands may be associated with a label such as a radioactive isotope or chelate thereof, dye (fluorescent or nonfluorescent,) stain, enzyme, metal, or any other substance capable of aiding a machine or a human eye from differentiating a cell expressing a marker from a cell not expressing a marker. Additionally, expression may be assessed by monomeric or multimeric ligands associated with substances capable of killing the cell. Such substances include protein or small molecule toxins, cytokines, pro-apoptotic substances, pore forming substances, radioactive isotopes, or any other substance capable of killing a cell.

Positive expression encompasses any difference between a cell expressing markers and a cell that does not express one or more of the markers. The exact nature of positive expression varies by the method, but is well known to those skilled in the art of practicing a particular method. Positive expression may be assessed by a detector, an instrument containing a detector, or by aided or unaided human eye. Examples include but are not limited to specific staining of cells expressing a target in an IHC slide, binding of RNA from a sample to a microarray and detection of binding through the use of said microarray, a particular rate of dye incorporation in real-time RTPCR measured in ΔCt or alternatively in the number of PCR cycles necessary to reach a particular optical density at a wavelength at which a double stranded DNA binding dye (e.g. SYBR Green) incorporates, through release of label from a previously labeled reporter probe used in a real-time RTPCR reaction, detection of fluorescence on a cell expressing a target by a flow cytometer, the presence of radiolabeled bands on film in a Northern blot, detection of labeled blocked RNA by RNAse protection assay, cell death measured by apoptotic markers, cell death measured by shrinkage of a tumor, or any other signal for the expression of a marker in existence now or yet to be developed. In some aspects of the invention, positive expression is a sufficient level of expression to correlate with a particular response such as susceptibility to cancer recurrence.

In some aspects of the invention, reduced expression constitutes no detectable expression. However, the concept of reduced expression further encompasses insufficient expression to reach or exceed a threshold, cutoff, or level that has been previously shown to result in a particular cellular or physiological response. Reduced expression may include similar expression relative to a control that has been previously determined not to express the marker(s) or similar expression to a control that has been previously determined not to exhibit the response. In this case, even though expression may be detectable, it still constitutes reduced expression. In some aspects of the invention, an expression level of a marker in a control known to have a reduced or increase risk of recurrence is predetermined and expression similar to that level is correlated with reduced or increase risk of recurrence. Increased or reduced expression includes expression that is 75% 50%, 25%, 10%, 5%, 1%, 0.1%, greater or less of that of a control cell or a median level of expression in a population. Reduced expression may also include greater than or less than 1×10⁻⁵ greater or less expression normalized to the expression of a housekeeping gene.

The invention contemplates assessing the expression of the marker(s) in any biological sample from which the expression may be assessed. One skilled in the art would know to select a particular biological sample and how to collect said sample depending upon the marker that is being assessed. Examples of sources of samples include but are not limited to biopsy or other in vivo or ex vivo analysis of prostate, breast, skin, muscle, fascia, brain, endometrium, lung, head and neck, pancreas, small intestine, blood, liver, testes, ovaries, colon, skin, stomach, esophagus, spleen, lymph node, bone marrow, kidney, placenta, or fetus. In some aspects of the invention, the sample comprises a fluid sample, such as peripheral blood, lymph fluid, ascites, serous fluid, pleural effusion, sputum, cerebrospinal fluid, amniotic fluid, lacrimal fluid, stool, or urine. In one aspect of the invention, the sample comprises primary or metastatic NSCLC cells. In another, the sample comprises sputum. In another aspect of the invention, the sample comprises blood.

Assessing the risk of a particular disease outcome includes the performing of any type of test, assay, examination, result, readout, or interpretation that correlates with an increased or decreased probability that an individual has had, currently has, or will develop a particular disease, disorder, symptom, syndrome, or any condition related to health or bodily state. Examples of disease outcomes include, but need not be limited to survival, death, progression of existing disease, remission of existing disease, initiation of onset of a disease in an otherwise disease-free subject, or the continued lack of disease in a subject in which there has been a remission of disease. Assessing the risk of a particular disease encompasses diagnosis in which the type of disease afflicting a subject is determined. Assessing the risk of a disease outcome also encompasses the concept of prognosis. A prognosis may be any assessment of the risk of disease outcome in an individual in which a particular disease has been diagnosed. Assessing the risk further encompasses prediction of therapeutic response in which a treatment regimen is chosen based on the assessment. Assessing the risk also encompasses a prediction of overall survival after diagnosis.

The sample in this method is preferably a biological sample from a subject. The term “sample” or “biological sample” is used in its broadest sense. Depending upon the embodiment of the invention, for example, a sample may comprise a bodily fluid including whole blood, serum, plasma, urine, saliva, cerebral spinal fluid, semen, vaginal fluid, pulmonary fluid, tears, perspiration, mucus and the like; an extract from a cell, chromosome, organelle, or membrane isolated from a cell; a cell; genomic DNA, RNA, or cDNA, in solution or bound to a substrate; a tissue; a tissue print, or any other material isolated in whole or in part from a living subject. Biological samples may also include sections of tissues such as biopsy and autopsy samples, and frozen sections taken for histologic purposes such as blood, plasma, serum, sputum, stool, tears, mucus, hair, skin, and the like. Biological samples also include explants and primary and/or transformed cell cultures derived from patient tissues.

The term “subject” is used in its broadest sense. In a preferred embodiment, the subject is a mammal. Non-limiting examples of mammals include humans, dogs, cats, horses, cows, sheep, goats, and pigs. Preferably, a subject includes any human or non-human mammal, including for example: a primate, cow, horse, pig, sheep, goat, dog, cat, or rodent, capable of developing cancer including human patients that are suspected of having cancer, that have been diagnosed with cancer, or that have a family history of cancer.

Some embodiments of the invention may include a method of comparing a marker in a sample relative to one or more control samples. A control may be any sample with a previously determined level of expression. A control may comprise material within the sample or material from sources other than the sample. Alternatively, the expression of a marker in a sample may be compared to a control that has a level of expression predetermined to signal or not signal a cellular or physiological characteristic. This level of expression may be derived from a single source of material including the sample itself or from a set of sources.

Cancer cells include any cells derived from a tumor, neoplasm, cancer, precancer, cell line, malignancy, or any other source of cells that have the potential to expand and grow to an unlimited degree. Cancer cells may be derived from naturally occurring sources or may be artificially created. Cancer cells may also be capable of invasion into other tissues and metastasis. Cancer cells further encompass any malignant cells that have invaded other tissues and/or metastasized. One or more cancer cells in the context of an organism may also be called a cancer, tumor, neoplasm, growth, malignancy, or any other term used in the art to describe cells in a cancerous state.

Examples of cancers that could serve as sources of cancer cells include solid tumors such as fibrosarcoma, myxosarcoma, liposarcoma, chondrosarcoma, osteogenic sarcoma, chordoma, angiosarcoma, endothelio sarcoma, lymphangiosarcoma, lymphangioendothelio sarcoma, synovioma, mesothelioma, Ewing's tumor, leiomyosarcoma, rhabdomyosarcoma, colon cancer, colorectal cancer, kidney cancer, pancreatic cancer, bone cancer, breast cancer, ovarian cancer, prostate cancer, esophageal cancer, stomach cancer, oral cancer, nasal cancer, throat cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, uterine cancer, testicular cancer, small cell lung carcinoma, bladder carcinoma, lung cancer, epithelial carcinoma, glioma, glioblastoma multiforme, astrocytoma, medulloblastoma, craniopharyngioma, ependymoma, pinealoma, hemangioblastoma, acoustic neuroma, oligodendroglioma, meningioma, skin cancer, melanoma, neuroblastoma, and retinoblastoma.

Additional cancers that may serve as sources of cancer cells include blood borne cancer, such as acute lymphoblastic leukemia (“ALL”), acute lymphoblastic B-cell leukemia, acute lymphoblastic T-cell leukemia, acute myeloblastic leukemia (“AML”), acute promyelocytic leukemia (“APL”), acute monoblastic leukemia, acute erythroleukemic leukemia, acute megakaryoblastic leukemia, acute myelomonocytic leukemia, acute nonlymphocyctic leukemia, acute undifferentiated leukemia, chronic myelocytic leukemia (“CML”), chronic lymphocytic leukemia (“CLL”), hairy cell leukemia, multiple myeloma, lymphoblastic leukemia, myelogenous leukemia, lymphocytic leukemia, myelocytic leukemia, Hodgkin's disease, non-Hodgkin's Lymphoma, Waldenstrom's macroglobulinemia, Heavy chain disease, and Polycythemia vera.

In some aspects of the invention, the cancer cells are derived from NSCLC, which comprises any carcinoma derived from lung tissues that does not include small cell lung cancers. Examples of non-small cell lung cancers include adenocarcinomas, large cell carcinomas, and squamous cell carcinomas of the lung.

The pathologic stages of non-small cell lung cancer include, but are not limited to the following: in the occult or hidden stage, cancer cells may be found in sputum, but no tumor can be found in the lung by bronchoscopy or other imaging. In Stage 0, also called carcinoma in situ, abnormal cells are found in the innermost lining of the lung. Such abnormal cells are precancerous and may or may not become malignant and spread into nearby tissue.

In Stage I, a cancer has developed. There are two substages to stage 1. In Stage IA, the tumor presents only in the lung only and is 3 centimeters or smaller. For the disease to be considered stage 1B, it will have one or more of the following traits: the tumor is larger than 3 centimeters, the cancer has spread to the main bronchus of the lung, and is at least 2 centimeters from the carina, the cancer has spread to the innermost layer of the membrane that covers the lungs, or the tumor partly blocks the bronchus or bronchioles and part of the lung has collapsed or developed pneumonitis (inflammation of the lung).

Similarly, there are two substages to Stage II. In Stage IIA, the tumor is 3 centimeters or smaller and cancer has spread to nearby lymph nodes on the same side of the chest as the tumor. For the disease to be considered, Stage IIB, the cancer has spread to nearby lymph nodes on the same side of the chest as the tumor and it will have one or more of the following traits: the tumor is larger than 3 centimeters, the cancer has spread to the main bronchus of the lung and is 2 centimeters or more from the carina, the cancer has spread to the innermost layer of the membrane that covers the lungs, or the tumor partly blocks the bronchus or bronchioles and part of the lung has collapsed or developed pneumonitis (inflammation of the lung). Alternatively, the disease may be classified as Stage 2B if the cancer has not spread to the lymph nodes and it displays one or more of the following traits: cancer has spread to the chest wall, or the diaphragm, or the pleura between the lungs, or membranes surrounding the heart, the cancer has spread to the main bronchus of the lung and is no more than 2 centimeters from the carina, but has not spread to the trachea, cancer blocks the bronchus or bronchioles and the whole lung has collapsed or developed pneumonitis (inflammation of the lung). Stage III is also divided into two substages.

In stage IIIA, cancer has spread to lymph nodes on the same side of the chest as the tumor and it displays one or more of the following traits: cancer has spread to the main bronchus, the chest wall, the diaphragm, the pleura around the lungs, or the membrane around the heart, but has not spread to the trachea, or part or all of the lung may have collapsed or developed pneumonitis (inflammation of the lung). In stage IIIB, the tumor has spread to one or more of the following: lymph nodes above the collarbone or in the opposite side of the chest from the tumor, to the heart, to major blood vessels that lead to or from the heart, to the chest wall, to the diaphragm, to the trachea, to the esophagus, to the sternum or spine, to more than one area in the same lobe of the lung, or to the fluid of the pleural cavity surrounding the lung.

In stage IV, cancer may have spread to lymph nodes and has spread to another lobe of the lung or to other parts of the body, such as the brain, liver, adrenal glands, kidneys, or bone.

The present invention further provides kits to be used in assessing the expression of a marker in a subject to assess the risk of developing disease, diagnosing the subject as having a stage of the disease, or determining to which stage the disease has progressed. Kits include any combination of components that facilitates the performance of an assay. A kit that facilitates assessing the expression of the markers may include suitable nucleic acid-based and immunological reagents as well as suitable buffers, control reagents, and printed protocols.

Kits that facilitate nucleic acid based methods may further include one or more of the following: specific nucleic acids such as oligonucleotides, labeling reagents, enzymes including PCR amplification reagents such as Taq or Pfu, reverse transcriptase, or other, and/or reagents that facilitate hybridization, as previously described.

In some aspects of the invention, a probe may be affixed to a solid substrate. In other aspects of the invention, the sample may be affixed to a solid substrate. A probe or sample may be covalently bound to the substrate or it may be bound by some non-covalent interaction including electrostatic, hydrophobic, hydrogen bonding, Van Der Waals, magnetic, or any other interaction by which a probe such as an oligonucleotide probe may be attached to a substrate while maintaining its ability to recognize the allele to which it has specificity. A substrate may be any solid or semi-solid material onto which a probe may be affixed, attached or printed, either singly or in the formation of a microarray. Examples of substrate materials include but are not limited to polyvinyl, polystyrene, polypropylene, polyester or any other plastic, glass, silicon dioxide or other silanes, hydrogels, gold, platinum, microbeads, micelles and other lipid formations, nitrocellulose, or nylon membranes. The substrate may take any form, including a spherical bead or flat surface. For example, the probe may be bound to a substrate in the case of an array. The sample may be bound to a substrate in the case of a Southern Blot.

Some embodiments of the invention may include the administration of one or more pharmaceutical compositions to a subject that has been diagnosed with cancer. Such pharmaceutical compositions may take any physical form necessary depending on a number of factors including the desired method of administration and the physicochemical and stereochemical form taken by the compound or pharmaceutically acceptable salts of the compound. Such physical forms include a solid, liquid, gas, sol, gel, aerosol, or any other physical form now known or yet to be disclosed.

In some aspects of the invention, the pharmaceutical compositions can comprise one or more compounds or products that are capable of treating a subject with NSCLC. In some embodiments, the pharmaceutical compositions may comprise or include one or more compounds that are capable of affecting, augmenting, and/or inhibiting one or more of the markers. The pharmaceutical compositions may comprise one or more compounds that are capable of inhibiting one or more of the markers. For example, the pharmaceutical composition may comprise one or more compounds that are capable of reducing expression of one or more of the markers. In some aspects, the one or more compounds can reduce the transcription, translation, and/or post-translational processes associated generally or specifically with one or more of the markers.

For example, in some embodiments, a first pharmaceutical composition may comprise one or more compounds that are capable of inhibiting the expression and/or function of Mcl-1 and/or Fn14. In these aspects, the one or more compounds may comprise siRNA, shRNA, antibodies, or other molecules that are capable of inhibiting the expression and/or function of Mcl-1 and/or Fn14. As used herein, “inhibit” or “inhibiting” may refer to a complete or partial reduction in expression (translational, transcriptional, post-translational, etc.) or complete or partial reduction in function of one or more of the markers.

By way of example only, in some embodiments, the markers may comprise Mcl-1 and/or Fn14 and the one or more compounds in the first pharmaceutical composition may be capable of affecting Mcl-1 and/or Fn14. In some aspects, the Mcl-1 inhibitors may comprise compounds, small molecules, antibodies, etc. including but not limited to, TW-37, EU-5148, UMI-77, MIM1, ABT-263, maritoclax, etc. Additional Mcl-1 inhibitors can be found in the following publications (all of which are incorporated by reference in their entirety for all purposes): A. Frieberg et al., Discovery of Potent Myeloid Cell Leukemia 1 (Mcl 1) Inhibitors Using Fragment Based Methods and Structure Based Design, 56 J. Med Chem 15 (2013); J. Belmar & S. Fesik, Small Molecule Mcl-1 Inhibitors for the Treatment of Cancer, Pharmacology and Therapeutics (2014); and B. A. Quinn et al., Targeting Mcl-1 For The Therapy of Cancer, 20 Expert Opin Investig Drugs 1397 (2011). Moreover, in some aspects, Fn14 inhibitors may include siRNA, shRNA, antibodies, or other molecules are now known or may be discovered in the future, such as those disclosed in U.S. patent application Ser. No. 14/327,448, which is hereby incorporated by reference for any purposes. In some aspects, the one or more compounds, siRNA, shRNA, antibodies, or other molecules may be capable of affecting Mcl-1 and/or Fn14 via the augmentation of the ligands for these molecules. For example, the pharmaceutical composition may comprise a compound that inhibits binding of the ligand to the target (e.g., Mcl-1 or Fn14) or the first pharmaceutical composition may comprise a compound, such as an antibody that binds to a ligand and depletes the ligand from the local or systemic environment.

The current invention may also include the administration of a second treatment to the subject. This second treatment may have the same or a similar molecular target as the marker or it may act upstream or downstream of the molecular target of the compound with regard to one or more biochemical pathways. Moreover, in some aspects, the second treatment may comprise a second pharmaceutical composition, which can comprise other chemotherapeutic compounds, such as a platinum-derived pharmaceutical (e.g., cisplatin, carboplatin, etc.), paclitaxel, pemetrexed, bevacizumab, etc. In addition, the second treatment can comprise other therapies, such as radiation.

In addition, in some aspects, one or more treatments can be provided in the event of the detection of one or more of the markers. By way of example only, in some aspects, detection of increased expression of one or more of the markers (e.g., Mcl-1 and/or Fn14) can be indicative of a negative prognosis (e.g., indicative of the fact that the cancer is likely to metastasize). In these situations, some embodiments of the invention comprise the administration of one or more prophylactic treatments (e.g., radiation) to reduce the likelihood of metastasis and/or reduce the impact of the metastases (e.g., prophylactic cranial irradiation).

For example, the first and/or second pharmaceutical compositions may include one or more compounds that are believe to function as a treatment for one or more types of cancers. Cancer therapies that can be identified as candidate treatments by the methods of the invention include without limitation: 13-cis-Retinoic Acid, 2-CdA, 2-Chlorodeoxyadenosine, 5-Azacitidine, 5-Fluorouracil, 5-FU, 6-Mercaptopurine, 6-MP, 6-TG, 6-Thioguanine, Abraxane, Accutane®, Actinomycin-D, Adriamycin®, Adrucil®, Afinitor®, Agrylin®, Ala-Cort®, Aldesleukin, Alemtuzumab, ALIMTA, Alitretinoin, Alkaban-AQ®, Alkeran®, All-transretinoic Acid, Alpha Interferon, Altretamine, Amethopterin, Amifostine, Aminoglutethimide, Anagrelide, Anandron®, Anastrozole, Arabinosylcytosine, Ara-C, Aranesp®, Aredia®, Arimidex®, Aromasin®, Arranon®, Arsenic Trioxide, Asparaginase, ATRA, Avastin®, Azacitidine, BCG, BCNU, Bendamustine, Bevacizumab, Bexarotene, BEXXAR®, Bicalutamide, BiCNU, Blenoxane®, Bleomycin, Bortezomib, Busulfan, Busulfex®, C225, Calcium Leucovorin, Campath®, Camptosar®, Camptothecin-11, Capecitabine, Carac™, Carboplatin, Carmustine, Carmustine Wafer, Casodex®, CC-5013, CCI-779, CCNU, CDDP, CeeNU, Cerubidine®, Cetuximab, Chlorambucil, Cisplatin, Citrovorum Factor, Cladribine, Cortisone, Cosmegen®, CPT-11, Cyclophosphamide, Cytadren®, Cytarabine, Cytarabine Liposomal, Cytosar-U®, Cytoxan®, Dacarbazine, Dacogen, Dactinomycin, Darbepoetin Alfa, Dasatinib, Daunomycin Daunorubicin, Daunorubicin Hydrochloride, Daunorubicin Liposomal, DaunoXome®, Decadron, Decitabine, Delta-Cortef®, Deltasone®, Denileukin, Diftitox, DepoCyt™, Dexamethasone, Dexamethasone Acetate Dexamethasone Sodium Phosphate, Dexasone, Dexrazoxane, DHAD, DIC, Diodex Docetaxel, Doxil®, Doxorubicin, Doxorubicin Liposomal, Droxia™, DTIC, DTIC-Dome®, Duralone®, Efudex®, Eligard™, Ellence™, Eloxatin™, Elspar®, Emcyt®, Epirubicin, Epoetin Alfa, Erbitux, Erlotinib, Erwinia L-asparaginase, Estramustine, Ethyol Etopophos®, Etoposide, Etoposide Phosphate, Eulexin®, Everolimus, Evista®, Exemestane, Fareston®, Faslodex®, Femara®, Filgrastim, Floxuridine, Fludara®, Fludarabine, Fluoroplex®, Fluorouracil, Fluorouracil (cream), Fluoxymesterone, Flutamide, Folinic Acid, FUDR®, Fulvestrant, G-CSF, Gefitinib, Gemcitabine, Gemtuzumab ozogamicin, Gemzar, Gleevec™, Gliadel® Wafer, GM-CSF, Goserelin, Granulocyte-Colony Stimulating Factor, Granulocyte Macrophage Colony Stimulating Factor, Halotestin®, Herceptin®, Hexadrol, Hexalen®, Hexamethylmelamine, HMM, Hycamtin®, Hydrea®, Hydrocort Acetate®, Hydrocortisone, Hydrocortisone Sodium Phosphate, Hydrocortisone Sodium Succinate, Hydrocortone Phosphate, Hydroxyurea, Ibritumomab, Ibritumomab, Tiuxetan, Idamycin®, Idarubicin, Ifex®, IFN-alpha, Ifosfamide, IL-11, IL-2, Imatinib mesylate, Imidazole Carboxamide, Interferon alfa, Interferon Alfa-2b (PEG Conjugate), Interleukin-2, Interleukin-11, Intron A® (interferon alfa-2b), Iressa®, Irinotecan, Isotretinoin, Ixabepilone, Ixempra™, Kidrolase (t), Lanacort®, Lapatinib, L-asparaginase, LCR, Lenalidomide, Letrozole, Leucovorin, Leukeran, Leukine™, Leuprolide, Leurocristine, Leustatin™, Liposomal Ara-C Liquid Pred®, Lomustine, L-PAM, L-Sarcolysin, Lupron®, Lupron Depot®, Matulane®, Maxidex, Mechlorethamine, Mechlorethamine Hydrochloride, Medralone®, Medrol®, Megace®, Megestrol, Megestrol Acetate, Melphalan, Mercaptopurine, Mesna, Mesnex™, Methotrexate, Methotrexate Sodium, Methylprednisolone, Meticorten®, Mitomycin, Mitomycin-C, Mitoxantrone, M-Prednisol®, MTC, MTX, Mustargen®, Mustine, Mutamycin®, Myleran®, Mylocel™, Mylotarg®, Navelbine®, Nelarabine, Neosar®, Neulasta™, Neumega®, Neupogen®, Nexavar®, Nilandron®, Nilutamide, Nipent®, Nitrogen Mustard, Novaldex®, Novantrone®, Octreotide, Octreotide acetate, Oncospar®, Oncovin®, Ontak®, Onxal™, Oprevelkin, Orapred®, Orasone®, Oxaliplatin, Paclitaxel, Paclitaxel Protein-bound, Pamidronate, Panitumumab, Panretin®, Paraplatin®, Pediapred®, PEG Interferon, Pegaspargase, Pegfilgrastim, PEG-INTRON™, PEG-L-asparaginase, PEMETREXED, Pentostatin, Phenylalanine Mustard, Platinol®, Platinol-AQ®, Prednisolone, Prednisone, Prelone®, Procarbazine, PROCRIT®, Proleukin®, Prolifeprospan 20 with Carmustine Implant, Purinethol®, Raloxifene, Revlimid®, Rheumatrex®, Rituxan®, Rituximab, Roferon-A® (Interferon Alfa-2a), Rubex®, Rubidomycin hydrochloride, Sandostatin®, Sandostatin LAR®, Sargramostim, Solu-Cortef®, Solu-Medrol®, Sorafenib, SPRYCEL™, STI-571, Streptozocin, SU11248, Sunitinib, Sutent®, Tamoxifen, Tarceva®, Targretin®, Taxol®, Taxotere®, Temodar®, Temozolomide, Temsirolimus, Teniposide, TESPA, Thalidomide, Thalomid®, TheraCys®, Thioguanine, Thioguanine Tabloid®, Thiophosphoamide, Thioplex®, Thiotepa, TICE®, Toposar®, Topotecan, Toremifene, Torisel®, Tositumomab, Trastuzumab, Treanda®, Tretinoin, Trexall™, Trisenox®, TSPA, TYKERB®, VCR, Vectibix™′ Velban®, Velcade®, VePesid®, Vesanoid®, Viadur™, Vidaza®, Vinblastine, Vinblastine Sulfate, Vincasar Pfs®, Vincristine, Vinorelbine, Vinorelbine tartrate, VLB, VM-26, Vorinostat, VP-16, Vumon®, Xeloda®, Zanosar®, Zevalin™, Zinecard®, Zoladex®, Zoledronic acid, Zolinza, Zometa®, and any appropriate combinations thereof.

The candidate treatments identified according to the subject methods can be chosen from the class of therapeutic agents identified as Anthracyclines and related substances, Anti-androgens, Anti-estrogens, Antigrowth hormones (e.g., Somatostatin analogs), Combination therapy (e.g., vincristine, bcnu, melphalan, cyclophosphamide, prednisone (VBMCP)), DNA methyltransferase inhibitors, Endocrine therapy—Enzyme inhibitor, Endocrine therapy—other hormone antagonists and related agents, Folic acid analogs (e.g., methotrexate), Folic acid analogs (e.g., pemetrexed), Gonadotropin releasing hormone analogs, Gonadotropin-releasing hormones, Monoclonal antibodies (EGFR-Targeted—e.g., panitumumab, cetuximab), Monoclonal antibodies (Her2-Targeted—e.g., trastuzumab), Monoclonal antibodies (Multi-Targeted—e.g., alemtuzumab), Other alkylating agents, Other antineoplastic agents (e.g., asparaginase), Other antineoplastic agents (e.g., ATRA), Other antineoplastic agents (e.g., bexarotene), Other antineoplastic agents (e.g., celecoxib), Other antineoplastic agents (e.g., gemcitabine), Other antineoplastic agents (e.g., hydroxyurea), Other antineoplastic agents (e.g., irinotecan, topotecan), Other antineoplastic agents (e.g., pentostatin), Other cytotoxic antibiotics, Platinum compounds, Podophyllotoxin derivatives (e.g., etoposide), Progestogens, Protein kinase inhibitors (EGFR-Targeted), Protein kinase inhibitors (Her2 targeted therapy—e.g., lapatinib), Pyrimidine analogs (e.g., cytarabine), Pyrimidine analogs (e.g., fluoropyrimidines), Salicylic acid and derivatives (e.g., aspirin), Src-family protein tyrosine kinase inhibitors (e.g., dasatinib), Taxanes, Taxanes (e.g., nab-paclitaxel), Vinca Alkaloids and analogs, Vitamin D and analogs, Monoclonal antibodies (Multi-Targeted—e.g., bevacizumab), Protein kinase inhibitors (e.g., imatinib, sorafenib, sunitinib).

In some embodiments, the candidate treatments identified according to the subject methods are chosen from at least the groups of treatments consisting of 5-fluorouracil, abarelix, alemtuzumab, aminoglutethimide, anastrozole, asparaginase, aspirin, ATRA, azacitidine, bevacizumab, bexarotene, bicalutamide, calcitriol, capecitabine, carboplatin, celecoxib, cetuximab, chemotherapy, cholecalciferol, cisplatin, cytarabine, dasatinib, daunorubicin, decitabine, doxorubicin, epirubicin, erlotinib, etoposide, exemestane, flutamide, fulvestrant, gefitinib, gemcitabine, gonadorelin, goserelin, hydroxyurea, imatinib, irinotecan, lapatinib, letrozole, leuprolide, liposomal-doxorubicin, medroxyprogesterone, megestrol, megestrol acetate, methotrexate, mitomycin, nab-paclitaxel, octreotide, oxaliplatin, paclitaxel, panitumumab, pegaspargase, pemetrexed, pentostatin, sorafenib, sunitinib, tamoxifen, Taxanes, temozolomide, toremifene, trastuzumab, VBMCP, and vincristine.

The concept of a pharmaceutical composition encompasses a compound or a pharmaceutically acceptable salt thereof with or without any other additive. The physical form of the invention may affect the route of administration and one skilled in the art would know to choose a route of administration that takes into consideration both the physical form of the compound and the disorder to be treated. Pharmaceutical compositions that include the compound may be prepared using methodology well known in the pharmaceutical art. A pharmaceutical composition that includes the disclosed compound may include a second effective compound of a distinct chemical formula from the disclosed compound. This second effective compound may have the same or a similar molecular target as the target or it may act upstream or downstream of the molecular target of the compound with regard to one or more biochemical pathways.

Pharmaceutical compositions include materials capable of modifying the physical form of a dosage unit. In one nonlimiting example, the composition includes a material that forms a coating that contains the compound. Materials that may be used in a coating, include, for example, sugar, shellac, gelatin, or any other inert coating agent.

Pharmaceutical compositions including the disclosed compound may be prepared as a gas or aerosol. Aerosols encompass a variety of systems including colloids and pressurized packages. Delivery of a composition in this form may include propulsion of a pharmaceutical composition including the disclosed compound through use of liquefied gas or other compressed gas or by a suitable pump system. Aerosols may be delivered in single phase, bi-phasic, or multi-phasic systems.

In some aspects of the invention, the pharmaceutical composition including the disclosed compound is in the form of a solvate. Such solvates are produced by the dissolution of the disclosed compound in a pharmaceutically acceptable solvent. Pharmaceutically acceptable solvents include any mixtures of one or more solvents. Such solvents may include pyridine, chloroform, propan-1-ol, ethyl oleate, ethyl lactate, ethylene oxide, water, ethanol, and any other solvent that delivers a sufficient quantity of the disclosed compound to treat the indicated condition.

Pharmaceutical compositions may also include at least one pharmaceutically acceptable carrier. Carriers include any substance that may be administered with the disclosed compound with the intended purpose of facilitating, assisting, or helping the administration or other delivery of the compound. Carriers include any liquid, solid, semisolid, gel, aerosol or anything else that may be combined with the disclosed compound to aid in its administration. Examples include diluents, adjuvants, excipients, water, and oils (including petroleum, animal, vegetable or synthetic oils.) Such carriers include particulates such as a tablet or powder, liquids such as oral syrup or injectable liquid, and inhalable aerosols. Further examples include saline, gum acacia, gelatin, starch paste, talc, keratin, colloidal silica, and urea. Such carriers may further include binders such as ethyl cellulose, carboxymethylcellulose, microcrystalline cellulose, or gelatin; excipients such as starch, lactose or dextrins; disintegrating agents such as alginic add, sodium alginate, Primogel, and corn starch; lubricants such as magnesium stearate or Sterotex; glidants such as colloidal silicon dioxide; sweetening agents such as sucrose or saccharin, a flavoring agent such as peppermint, methyl salicylate or orange flavoring, or coloring agents. Further examples of carriers include polyethylene glycol, cyclodextrin, oils, or any other similar liquid carrier that may be formulated into a capsule. Still further examples of carriers include sterile diluents such as water for injection, saline solution, physiological saline. Ringer's solution, isotonic sodium chloride, fixed oils such as synthetic mono or diglycerides, polyethylene glycols, glycerin, cyclodextrin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose, thickening agents, lubricating agents, and coloring agents.

The pharmaceutical composition may take any of a number of formulations depending on the physicochemical form of the composition and the type of administration. Such forms include solutions, suspensions, emulsions, tablets, pills, pellets, capsules, capsules including liquids, powders, sustained-release formulations, directed release formulations, lyophylates, suppositories, emulsions, aerosols, sprays, granules, powders, syrups, elixirs, or any other formulation now known or yet to be disclosed. Additional examples of suitable pharmaceutical carriers and formulations are well known in the art.

Methods of administration include, but are not limited to, oral administration and parenteral administration. Parenteral administration includes, but is not limited to intradermal, intramuscular, intraperitoneal, intravenous, subcutaneous, intranasal, epidural, sublingual, intranasal, intracerebral, iratraventricular, intrathecal, intravaginal, transdermal, rectal, by inhalation, or topically to the ears, nose, eyes, or skin. Other methods of administration include but are not limited to infusion techniques including infusion or bolus injection, by absorption through epithelial or mucocutaneous linings such as oral mucosa, rectal and intestinal mucosa. Compositions for parenteral administration may be enclosed in ampoule, a disposable syringe or a multiple-dose vial made of glass, plastic or other material.

Administration may be systemic or local. Local administration is administration of the disclosed compound to the area in need of treatment. Examples include local infusion during surgery; topical application, by local injection; by a catheter; by a suppository; or by an implant. Administration may be by direct injection into the central nervous system by any suitable route, including intraventricular and intrathecal injection. Intraventricular injection can be facilitated by an intraventricular catheter, for example, attached to a reservoir, such as an Ommaya reservoir. Pulmonary administration may be achieved by any of a number of methods known in the art. Examples include the use of an inhaler or nebulizer, formulation with an aerosolizing agent, or via perfusion in a fluorocarbon or synthetic pulmonary surfactant. Compounds may be delivered in the context of a vesicle such as a liposome or any other natural or synthetic vesicle. Additional examples of suitable modes of administration are well known in the art.

A pharmaceutical composition formulated to be administered by injection may be prepared by dissolving the disclosed compound with water so as to form a solution. In addition, a surfactant may be added to facilitate the formation of a homogeneous solution or suspension.

Surfactants include any complex capable of non-covalent interaction with the disclosed compound so as to facilitate dissolution or homogeneous suspension of the compound.

Pharmaceutical compositions may be prepared in a form that facilitates topical or transdermal administration. Such preparations may be in the form of a solution, emulsion, ointment, gel base, transdermal patch or iontophoresis device. Examples of bases used in such compositions include petrolatum, lanolin, polyethylene glycols, beeswax, mineral oil, diluents such as water and alcohol, and emulsifiers and stabilizers, thickening agents, or any other suitable base now known or yet to be disclosed.

Determination of an effective amount of the disclosed compound is within the capability of those skilled in the art, especially in light of the detailed disclosure provided herein. The effective amount of a pharmaceutical composition used to affect a particular purpose as well as its toxicity, excretion, and overall tolerance may be determined in vitro, or in vivo, by pharmaceutical and toxicological procedures either known now by those skilled in the art or by any similar method yet to be disclosed. One example is the in vitro determination of the IC₅₀ (half maximal inhibitory concentration) of the pharmaceutical composition in cell lines or target molecules. Another example is the in vivo determination of the LD₅₀ (lethal dose causing death in 50% of the tested animals) of the pharmaceutical composition. The exact techniques used in determining an effective amount will depend on factors such as the type and physical/chemical properties of the pharmaceutical composition, the property being tested, and whether the test is to be performed in vitro or in vivo. The determination of an effective amount of a pharmaceutical composition will be well known to one of skill in the art who will use data obtained from any tests in making that determination. Determination of an effective amount of disclosed compound for administration also includes the determination of an effective therapeutic amount and a pharmaceutically acceptable dose, including the formulation of an effective dose range for use in vivo, including in humans.

Treatment of a condition or disease is the practice of any method, process, or procedure with the intent of halting, inhibiting, slowing or reversing the progression of a disease, disorder or condition, substantially ameliorating clinical symptoms of a disease disorder or condition, or substantially preventing the appearance of clinical symptoms of a disease, disorder or condition, up to and including returning the diseased entity to its condition prior to the development of the disease. Generally, the effectiveness of treatment is determined by comparing treated groups with non-treated groups.

The addition of a therapeutically effective amount of a compound encompasses any method of dosing of a compound. Dosing of the disclosed compound may include single or multiple administrations of any of a number of pharmaceutical compositions that include the disclosed compound as an active ingredient. Examples include a single administration of a slow release composition, a course of treatment involving several treatments on a regular or irregular basis, multiple administrations for a period of time until a diminution of the disease state is achieved, preventative treatments applied prior to the instigation of symptoms, or any other dosing regimen known in the art or yet to be disclosed that one skilled in the art would recognize as a potentially effective regimen. A dosing regimen including the regularity of and mode of administration will be dependent on any of a number of factors including but not limited to the subject being treated; the severity of the condition; the manner of administration, the stage of disease development, the presence of one or more other conditions such as pregnancy, infancy, or the presence of one or more additional diseases; or any other factor now known or yet to be disclosed that affects the choice of the mode of administration, the dose to be administered and the time period over which the dose is administered.

Pharmaceutical compositions may be administered prior to, concurrently with, or after administration of additional or second pharmaceutical compositions. Concurrent administration means compositions are administered within about one minute of each other. If not administered concurrently, the additional or second pharmaceutical compositions may be administered a period of one or more minutes, hours, days, weeks, or months before or after the pharmaceutical composition that includes the currently disclosed compound. Alternatively, a combination of pharmaceutical compositions may be cyclically administered. Cycling therapy involves the administration of one or more pharmaceutical compositions for a period of time, followed by the administration of one or more different pharmaceutical compositions for a period of time and repeating this sequential administration. Cycling therapy may be used, for example, to reduce the development of resistance to one or more of the compositions, to avoid or reduce the side effects of one or more of the compositions, and/or to improve the efficacy of the treatment.

The invention further encompasses kits that facilitate the administration of the disclosed compound to a diseased entity. An example of such a kit includes one or more unit dosages of one or more active ingredients. The unit dosage would be enclosed in a preferably sterile container and would be comprised of the compound and a pharmaceutically acceptable carrier. In another aspect, the unit dosage would comprise one or more lyophilates of the compound. In this aspect of the invention, the kit may include another preferably sterile container enclosing a solution capable of dissolving the lyophilate. However, such a solution need not be included in the kit and may be obtained separately from the lyophilate. In another aspect, the kit may include one or more devices used in administrating the unit dosages or a pharmaceutical composition to be used in combination with the compound. Examples of such devices include, but are not limited to, a syringe, a drip bag, a patch or an enema. In some aspects of the invention, the device comprises the container that encloses the unit dosage. In another aspect, the kit may include one or more additional compounds for administration and administration instructions therefor.

Overall, some embodiments of the invention include systems and methods for the diagnosis of a condition, assessing the prognosis of the condition, and treating the condition. Particular embodiments comprise the use of one or more markers (e.g., Mcl-1 and/or Fn14) to diagnosis one or more forms of cancer and/or assessing the likely stage of progression of the cancer. For example, the one or more markers can be used to diagnose and/or assess the likely stage of the cancer. In some aspects, the cancer may comprise non-small cell lung cancer. Moreover, some embodiments of the invention may further provide treating a cancer, such as non-small cell lung cancer. For example, the method of treatment may comprise altering the expression of one or more markers (e.g., Mcl-1) and then treating the cancer using one or more treatment modalities, such as radiation-based therapy or chemotherapy. In particular, the expression of the marker may be reduced, which may sensitize the cancer cells to the one or more treatment modalities.

### Examples

### Materials and Methods

Cell Culture Conditions

Human lung adenocarcinoma cell lines H1975 and H2073 (ATCC, Manassas, Va.) were maintained in RPMI 1640 media (Invitrogen, Carlsbad, Calif.) plus 10% heat-inactivated fetal bovine serum (FBS) in a 37° C., 5% CO₂ atmosphere. SU-DHL10 (Deng J, Carlson N, Takeyama K, Dal Cin P, Shipp M, Letai A. BH3 profiling identifies three distinct classes of apoptotic blocks to predict response to ABT-737 and conventional chemotherapeutic agents. Cancer Cell. 2007; 12:171-85), Bcl-2 1863 and Mcl-1 1780 cell lines (Ryan J A, Brunelle J K, Letai A. Heightened mitochondrial priming is the basis for apoptotic hypersensitivity of CD4+CD8+ thymocytes. Proc Natl Acad Sci USA. 2010; 107:12895-900) were maintained in RPMI 1640 plus 10% FBS with 50 μM β-mercaptoethanol added to 1863 and 1780 cells. In all assays treated with TWEAK, cells were cultured in reduced serum (0.5% FBS) for 16 hours prior to stimulation with TWEAK at 100 ng/mL in RPMI+0.1% bovine serum albumin (BSA) for the indicated times.

Immunohistochemistry (IHC)

Protein expression by IHC was performed on a tissue microarray as previously described (Whitsett T G, Cheng E, Inge L, Asrani K, Jameson N M, Hostetter G, et al. Elevated expression of Fn14 in non-small cell lung cancer correlates with activated EGFR and promotes tumor cell migration and invasion. Am J Pathol. 2012; 181:111-20), and IHC analysis for Fn14 has also been previously described (Whitsett T G, Cheng E, Inge L, Asrani K, Jameson N M, Hostetter G, et al. Elevated expression of Fn14 in non-small cell lung cancer correlates with activated EGFR and promotes tumor cell migration and invasion. Am J Pathol. 2012; 181:111-20). Mcl-1 staining was performed using an antibody specific for the long form of Mcl-1 (Santa Cruz Biotechnology, Dallas, Tex.) (SEQ ID NO: 2). A scoring system for each chromophore comprised of staining intensity and extensiveness captured the outcome: 0, negative; 1, weak; 2 moderate; 3, strong. A two-sided Kendall's tau test was carried out on scores of Mcl-1 and Fn14 for samples in which both were evaluated and scored.

Antibodies, Reagents, and Immunoblotting

Mcl-1, Bcl-2, Bcl-xL, phospho-p65 (Serine residue 536), Bak, GAPDH, and cleaved-PARP antibodies were obtained from Cell Signaling Technology Inc. (Beverly, Mass.), and α-tubulin antibody was obtained from Millipore (San Diego, Calif.). Human recombinant TWEAK was purchased from PeproTech (Rock Hill, N.J.), and cisplatin was obtained from TZS Chemical via BIOTANG Inc. (Waltham, Mass.). The Mcl-1-specific inhibitor EU-5148 was kindly provided by Eutropics Pharmaceuticals (Cambridge, Mass.). ABT-737 was obtained from Selleck Chemicals (Houston, Tex.). Immunoblot analysis was performed as previously described (Whitsett T G, Cheng E, Inge L, Asrani K, Jameson N M, Hostetter G, et al. Elevated expression of Fn14 in non-small cell lung cancer correlates with activated EGFR and promotes tumor cell migration and invasion. Am J Pathol. 2012; 181:111-20; Tran N L, McDonough W S, Savitch B A, Sawyer T F, Winkles J A, Berens M E. The tumor necrosis factor-like weak inducer of apoptosis (TWEAK)-fibroblast growth factor-inducible 14 (Fn14) signaling system regulates glioma cell survival via NFkB pathway activation and BCL-XL/BCL-W expression. J Biol Chem. 2005; 280:3483-92).

Quantitative, Real-Time PCR (qPCR)

mRNA expression was determined by qPCR as previously described (Fortin Ensign S P, Mathews I T, Eschbacher J M, Loftus J C, Symons M H, Tran N L. The Src homology 3 domain-containing guanine nucleotide exchange factor is overexpressed in high-grade gliomas and promotes tumor necrosis factor-like weak inducer of apoptosis-fibroblast growth factor-inducible 14-induced cell migration and invasion via tumor necrosis factor receptor-associated factor 2. J Biol Chem. 2013; 288:21887-97). Briefly, total RNA was extracted from cell lines using the mirVana isolation kit (Ambion, Austin, Tex.) according to the manufacturer's directions. cDNA was synthesized from total RNA using SuperScript III First-Strand Synthesis SuperMix (Life Technology, Grand Island, N.Y.) according to the manufacturer's protocol.

Quantitative RT-PCR analyses of Mcl-1 (forward: 5′-GGACTGGCTAGTTAAACAAAGAGG-3′ (SEQ ID NO: 5); reverse: 5′-CTTATTAGATATGCCAAACCAGCTC-3′) (SEQ ID NO: 6), Bcl-xL (BCL2L1) (forward: 5′-GCTGAGTTACCGGCATCC-3 (SEQ ID NO: 7)′; reverse: 5′-TTCTGAAGGGAGAGAAAGAGATTC-3′ (SEQ ID NO: 8)) and histone H3.3 (forward: 5′-CCACTAACTTCTGATTCGC-3′ (SEQ ID NO: 9); reverse: 5′-GCGTGCTAGCTGGATGTCTT-3′ (SEQ ID NO: 10)) were carried out in triplicate in a 384-well plate using a LightCycler 480 (Roche Applied Sciences, Indianapolis, Ind.) and analyzed as previously described (Tran N L, McDonough W S, Donohue P J, Winkles J A, Berens T J, Ross K R, et al. The human Fn14 receptor gene is up-regulated in migrating glioma cells in vitro and overexpressed in advanced glial tumors. Am J Pathol. 2003; 162:1313-21).

Expression Plasmids and Transfection

Cytomegalovirus plasmid backbones (pCMV) containing genes for human wild type IκBα or IκBα with serine-to-alanine mutations at residues 32 and 36 were purchased from Addgene (Cambridge, Mass.) and transfected into the human adenocarcinoma cell lines using the Effectine Transfection Reagent (Qiagen, Valencia, Calif.) kit according to the manufacturer's protocol.

Small-Interfering RNA Preparation and Transfection

Small interfering RNA (siRNA) oligonucleotides specific for GL2 Luciferase were previously described (Chuang Y Y, Tran N L, Rusk N, Nakada M, Berens M E, Symons M. Role of synaptojanin 2 in glioma cell migration and invasion. Cancer Res. 2004; 64:8271-5). Validated siRNA sequences for Mcl-1 full-length transcripts (Mcl-1-1 and Mcl-1-2 target oligonucleotide sequences: 5′-CCCGCCGAATTCATTAATTTA-3′ (SEQ ID NO: 11), 5′-CCCTAGCAACCTAGCCAGAAA-3′ (SEQ ID NO: 12), respectively) and Bcl-xL sequence: 5′-CTGCTTGGGATAAAGATGCAA-3′ (SEQ ID NO: 13) were purchased from Qiagen. Transient siRNA transfection was carried out as previously described (Chuang Y Y, Tran N L, Rusk N, Nakada M, Berens M E, Symons M. Role of synaptojanin 2 in glioma cell migration and invasion. Cancer Res. 2004; 64:8271-5). All siRNA transfections were done at 20 nM siRNA using Lipofectamine RNAi MAX reagent (Invitrogen) and no cytotoxicity were observed 24 hours post-transfection. Maximum inhibition of protein levels was achieved approximately 72 hours post-transfection.

Clonogenic Assay

Observations of colony forming capacity following cytotoxic insult were performed as previously described (Franken N A, Rodermond H M, Stap J, Haveman J, van Bree C. Clonogenic assay of cells in vitro. Nat Protoc. 2006; 1:2315-9). Briefly, cells were transfected with either luciferase or Mcl-1 siRNA, or treated with EU-5148 at 7.5 μM for 24 hours, followed by serum-reduction (0.5% FBS) for 16 hours prior to the addition of TWEAK for 24 hours. For cisplatin treatment, cells were pre-treated with TWEAK for 4 hours prior to the addition of cisplatin (1 μM) for 20 hours. For radiation treatment, cells were pre-treated with TWEAK and incubated for 24 hours before exposure to 2Gy ionizing irradiation using an RS-200 (Rad Source, Suwanee, Ga.). Cells were then trypsinized, quantified, and equally dispersed in triplicate in 6-well cell culture dishes at 250 cells per well. Plates were incubated until colonies reached an approximate size of 50 cells (1-2 weeks) before being fixed briefly in a 10% (v/v) methanol 10% (v/v) glacial acetic acid solution, stained with a 0.5% (w/v) crystal violet solution and washed with deionized water. Apparent colonies were counted. All cell lines and treatments were run in triplicate. Statistical significance, defined as a p value <0.05, was determined by ANOVA analysis with Bonferroni posttests in Graphpad Prism.

Immunoprecipitation

For immunoprecipitation, cells were treated with TWEAK, 7.5 μM EU-5148, or TWEAK and EU-5148 simultaneously. After 24 hours of treatment, cells were lysed on ice for 10 min in a buffer containing 10 mM Tris-HCl (pH 7.4), 0.5% Nonidet P-40, 150 mM NaCl, 1 mM phenylmethylsulfonyl fluoride, 1 mM EDTA, 2 mM sodium orthovanadate, 20 mM sodium fluoride, 10 μg/ml aprotinin, and 10 μg/ml leupeptin. Equivalent amounts of protein (500 μg) were pre-cleared and immunoprecipitated from each lysates using Bak antibody as indicated or a control isotype-matched antibody and then washed with lysis buffer followed by TX-100 buffer (10 mM HEPES (pH 7.4), 150 mM NaCl, 2 mM EDTA, 2 mM EGTA, 20 mM sodium fluoride, and 0.5% Triton X-100). Immunoprecipitated samples were then reconstituted in 2×SDS buffer containing protease and phosphatase inhibitors and immunoblotted with the indicated antibodies as described.

PrestoBlue Assay

Cell killing was measured by PrestoBlue cell viability reagent after incubating cells with compound for 48 hours. EU-5148 and DMSO were diluted in serum-free RPMI 1640 media and dispensed to a cell culture-treated 384-well plate (Griener Bio-One). Cells in culture were counted and centrifuged, then resuspended in RPMI 1640, 10% FBS, 1% Penicillin-streptomycin. Cells were added to the plate containing drug dilutions (5,000 cells/well) and incubated at 37° C. for 48 hours. PrestoBlue cell viability reagent (Invitrogen) was added to the plate and fluorescence was measured after 1 hour at excitation/emission 535/595 nm. Cell killing curves were made in GraphPad Prism 5.

Cell Viability Assay

NSCLC cell viability was tested using the CellTiter-Glo Luminescent Cell Viability Assay Kit (Promega, Madison, Wis.) according to the manufacturer's instructions. Cells were treated with EU-5148 for 48 hours prior to luminescence reading performed on a Victor3 1420 Multilabel Counter (PerkinElmer, Waltham, Mass.). All cell lines and treatments were performed in duplicate.

Competitive Displacement ELISA Assay

To measure the binding activity of EU-5148 to Mcl-1 and Bcl-xL, a competitive displacement assay was performed using EU-5148, Mcl-1/GST, and Biotin-Bim. Biotinylated Bim peptide was first diluted to 0.018 μg/mL in Superblock blocking buffer (Pierce) and added to a streptavidin-coated plate for ˜2 hours. Meanwhile, EU-5148 was incubated at room temperature with 20 nM Mcl-1/GST or Bcl-xL in PBS for 2 hours in siliconized microcentrifuge tubes; DMSO was titrated in equivalent concentrations as a control. The strepatividin plate was washed 3× with PBST (0.05% Tween 20 in PBS) and the compound/protein solutions were transferred to the plate and incubated for 2 hours. After washing the plate 3 times with PBST, anti-GST/HRP was then diluted in PBST and incubated on the plate for 30 minutes. The plate was then washed 5 times with PBST and color reagents (R&D kit) were added to develop the plate. Stop solution (2N H2504) was added after 5 minutes of incubation with color reagent. Absorbance was measured at 450 nm and binding curves generated in GraphPad Prism 5.

Results

Mcl-1 is Over-Expressed in Human Primary NSCLC Tumors and Correlates with Fn14

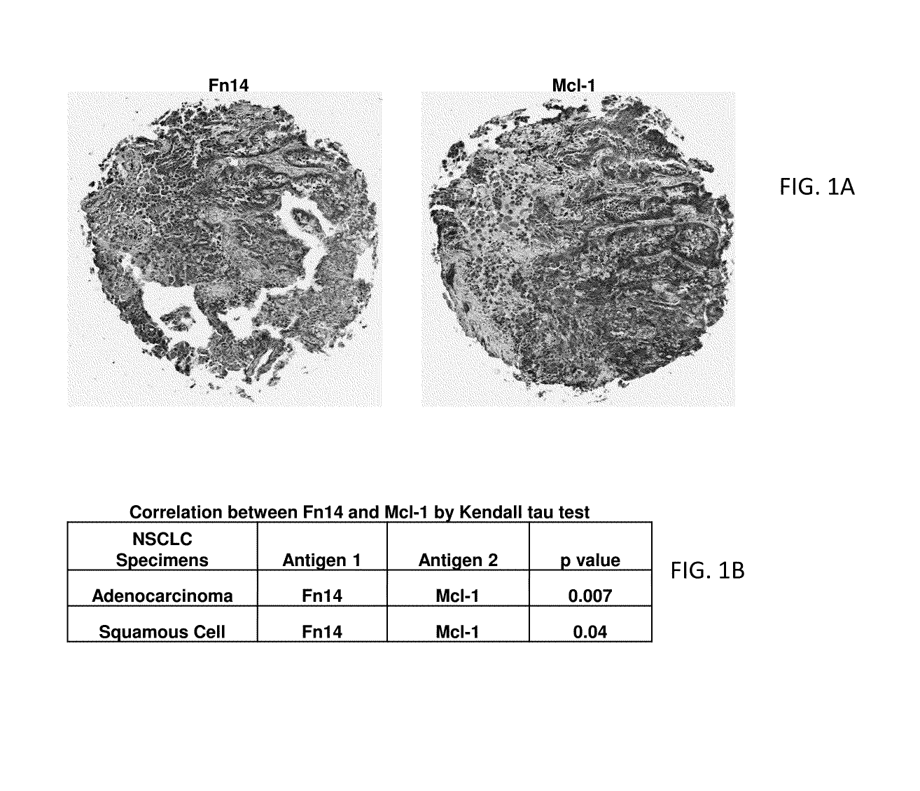

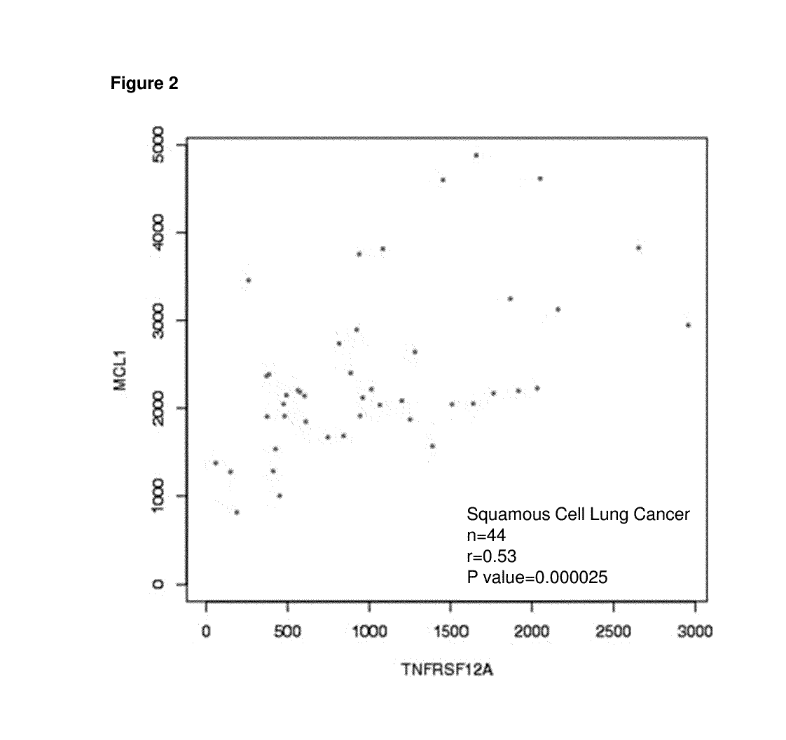

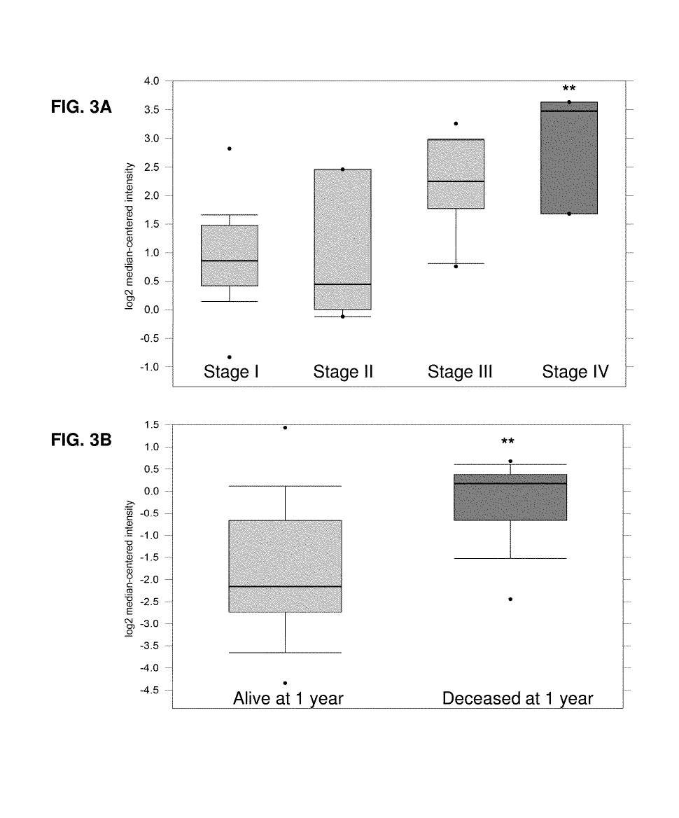

To determine the expression of MCL-1 protein in NSCLC, we utilized a NSCLC TMA as previously described. The protein expression of Mcl-1 was detected in the cytoplasm in the majority of NSCLC tumors (80% of adenocarcinomas and 58% of squamous cell carcinomas demonstrated moderate to strong protein levels of Mcl-1). Since the correlation of Fn14 expression and pro-survival Bcl-2 family members has been previously reported, we next examined the relationship between these two markers. In both adenocarcinomas and squamous cell carcinomas, the protein levels of Mcl-1 were significantly correlated with the protein levels of Fn14 (FIGS. 1A and 1B). Gene expression data further showed a significant correlation between Mcl-1 and Fn14 mRNA levels in squamous cell lung cancer specimens (FIG. 2). At the gene expression level, mRNA levels of Mcl-1 significantly correlate with increasing stage of lung adenocarcinomas (FIG. 3A) and with patient mortality at one year (FIG. 3B). Thus, Mcl-1 is highly expressed in primary NSCLC tumors, correlates with Fn14 expression, and is associated with poor patient prognosis.

TWEAK Stimulation Induces Mcl-1 and Other Bcl-2 Family Member Expression Through Activation of NF-κB

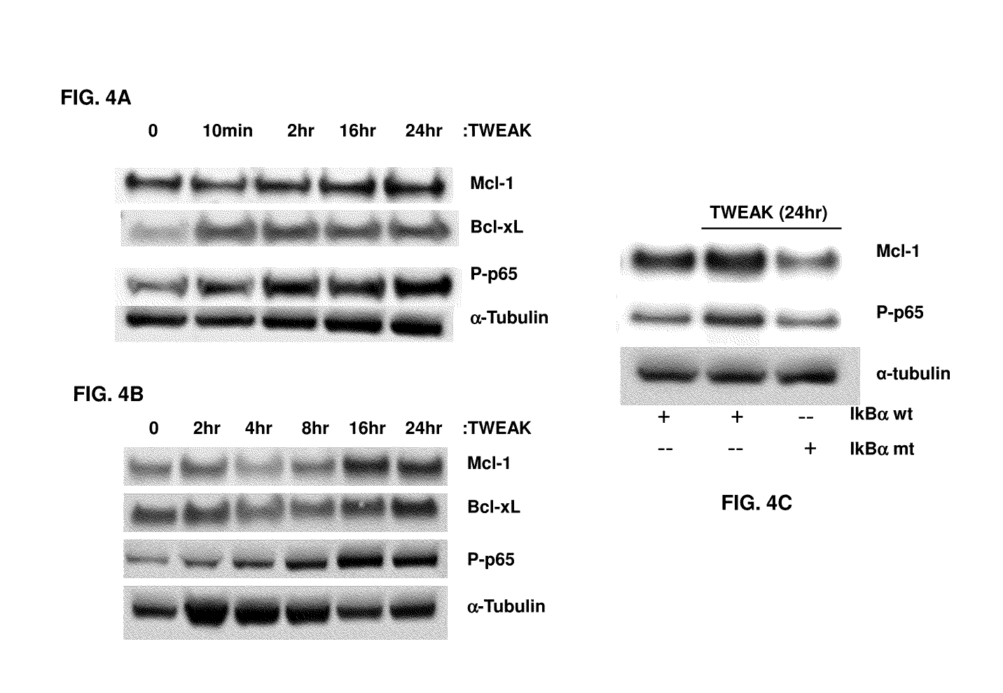

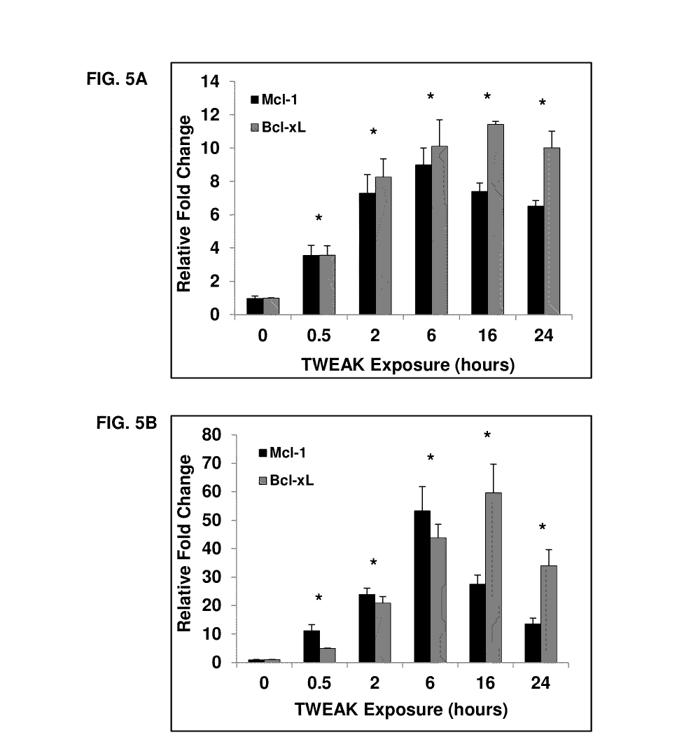

TWEAK promotes cancer cell survival through phosphorylation of the p65 subunit of NF-κB, leading to increased expression of pro-survival Bcl-2 family members Bcl-xL and Bcl-w. It was therefore investigated whether TWEAK stimulation induces pro-survival Bcl-2 members in NSCLC. In the adenocarcinoma cell lines H1975 and H2073, TWEAK treatment led to the clear induction of phosphorylated p65 (P-p65), with concomitant incremental increases in protein expression of Mcl-1 and Bcl-xL over time. (FIGS. 4A and 4B) The protein expression of Bcl-2 was also up-regulated following TWEAK exposure in H1975, but was not expressed in H2073. To confirm TWEAK-induced expression of Bcl-2 family members, we measured mRNA expression by qPCR. TWEAK treatment induced mRNA levels of Mcl-1 and Bcl-xL in both H1975 and H2073 cells as early as 30 minutes post-TWEAK with maximal expression at about 6 hours (10-fold increase in H1975 and 50-fold increase in H2073) (FIGS. 5A and 5B).

To determine whether TWEAK-induced Mcl-1 expression is dependent on NF-κB activation, H2073 cells were transfected with wild-type IκBα (IκBα-wt), or a mutated IκBα super-repressor (IκBα-mt) expressing plasmid, and levels of Mcl-1 following TWEAK exposure were assessed. The IκBα-mt is incapable of being phosphorylated and thus sequesters NF-κB in the cytoplasm. Mcl-1 protein expression increased with TWEAK treatment in IκBα-wt-expressing cells, but was inhibited in the presence of the IκBα-mt expression, in correlation with reduced p65 phosphorylation (FIG. 4C). These data suggest that NF-κB activation by TWEAK is necessary for the induction of Mcl-1 in NSCLC.

TWEAK-Induced NSCLC Cell Survival is Dependent on Mcl-1 Expression

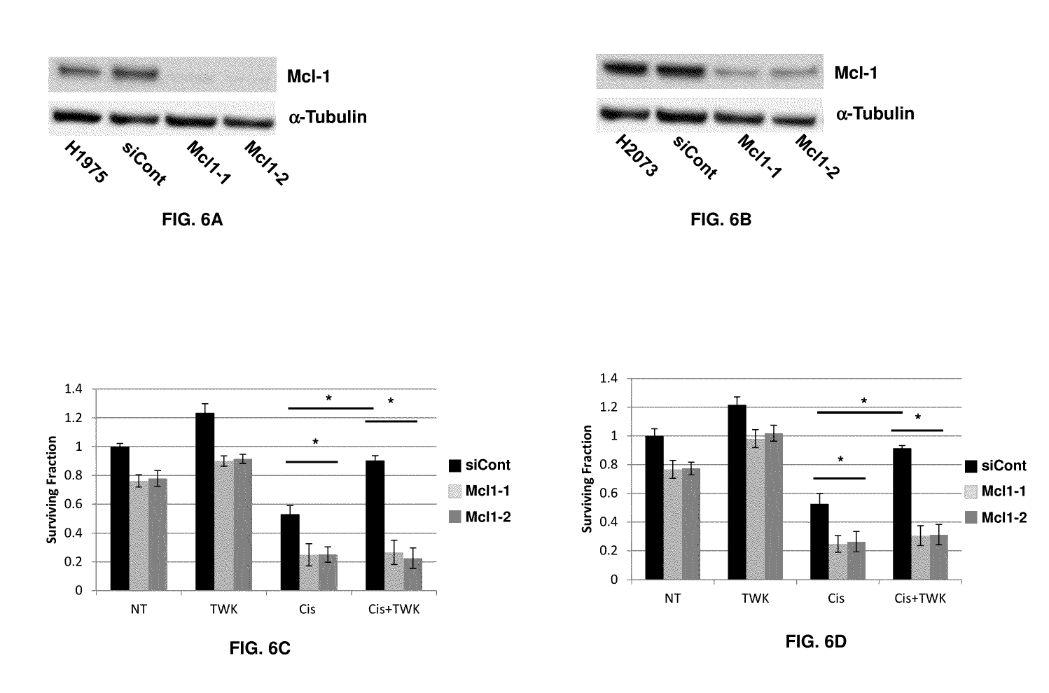

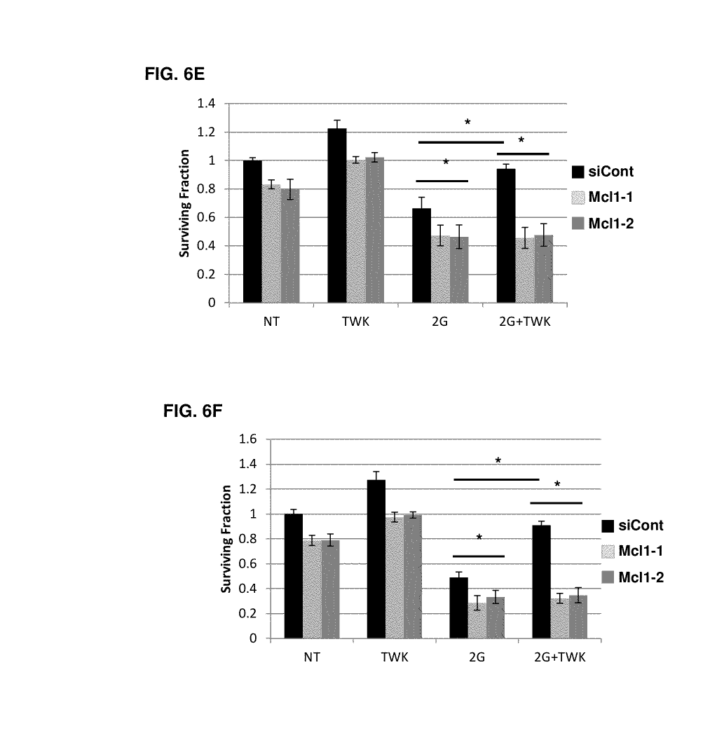

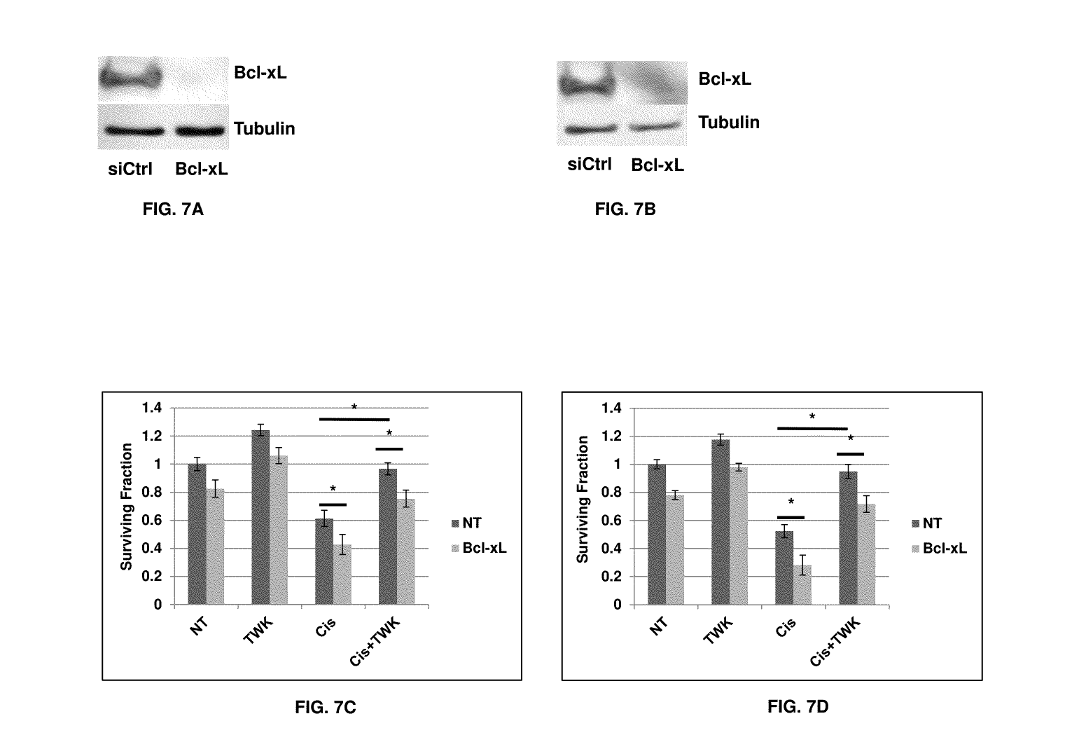

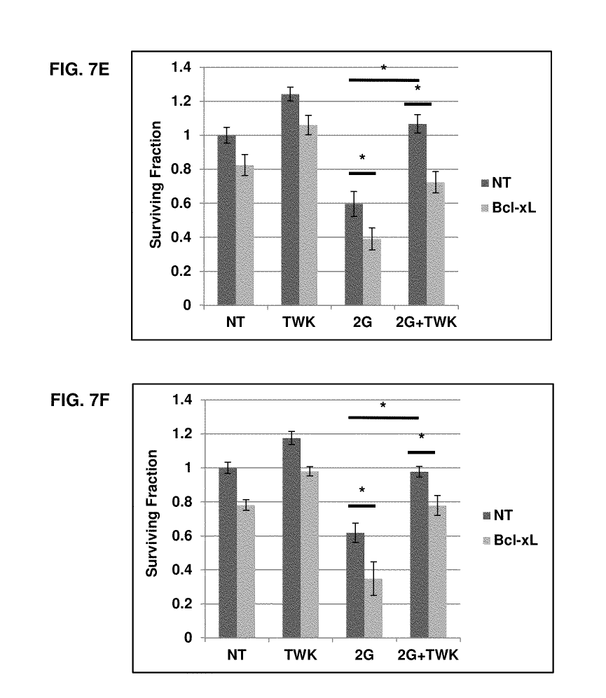

Characterizing the functional role of Mcl-1 in TWEAK-induced tumor cell survival was the next aim. The protein expression of Mcl-1 was depleted by targeted siRNA constructs in both H1975 and H2073 cells (FIGS. 6A and 6B, respectively). Cell survival was assessed by colony-formation assay. In both H1975 and H2073, exposure to ionizing radiation or cisplatin significantly reduced NSCLC cell survival (FIGS. 6C-6F). siRNA mediated depletion of Mcl-1 significantly enhanced sensitivity to either cisplatin or radiation compared to control cells expressing non-targeting siRNA oligonucleotides. TWEAK pre-treatment significantly attenuated the effects of either cisplatin or radiation, back to untreated surviving fractions. Depletion of Mcl-1 via siRNA oligonucleotides completely abrogated the protective effects observed with TWEAK pre-treatment. However, depletion of Bcl-xL could not fully rescue the TWEAK induced cell survival as seen with Mcl-1 depletion (FIGS. 7A-7B). Thus, TWEAK exposure may protect NSCLC cells from DNA-damaging therapies such as radiation and cisplatin; and this protective phenotype appears to be dependent on Mcl-1 function.

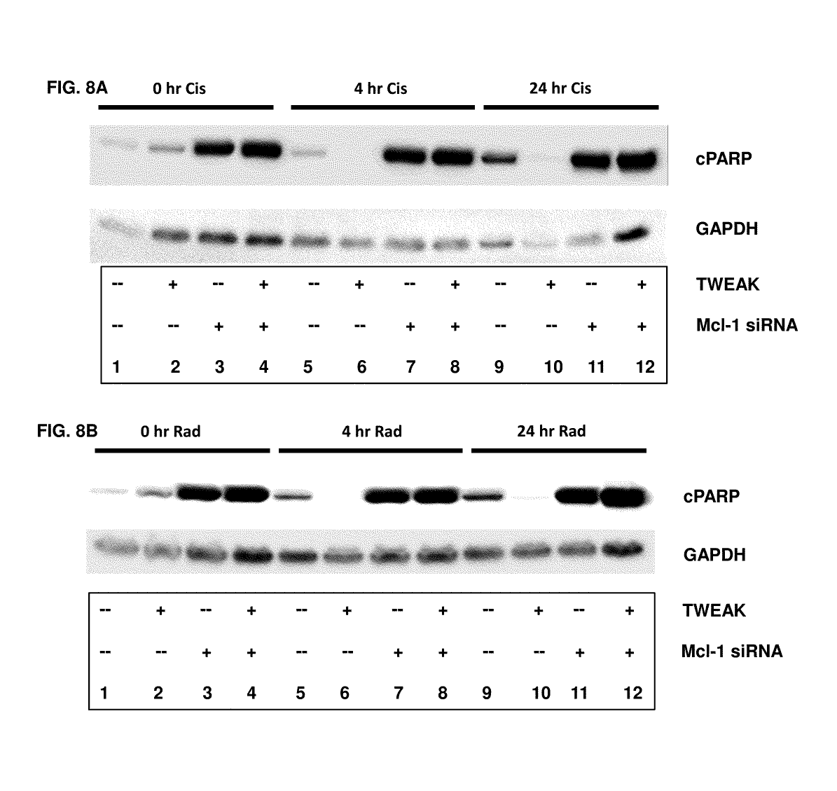

To confirm the effects of TWEAK signaling and the role of Mcl-1 in NSCLC cell survival, we assessed apoptosis through the induction of cleaved-PARP in H1975 cells exposed to cisplatin and radiation. FIG. 8 demonstrates that exposure to cisplatin (FIG. 8A) or radiation (FIG. 8B) induces protein expression of cleaved-PARP over time (lanes 5 and 9 compared to 1). Pretreatment with TWEAK completely abrogates the induction of cleaved-PARP (lanes 6 and 10 compared to lanes 5 and 9). The depletion of Mcl-1 through siRNA results in enhanced induction of cleaved-PARP compared to cisplatin or radiation alone, an enhanced sensitivity that was not affected by TWEAK exposure.

Mcl-1 Pharmacological Inhibitor EU-5148 Decreases NSCLC Cell Survival

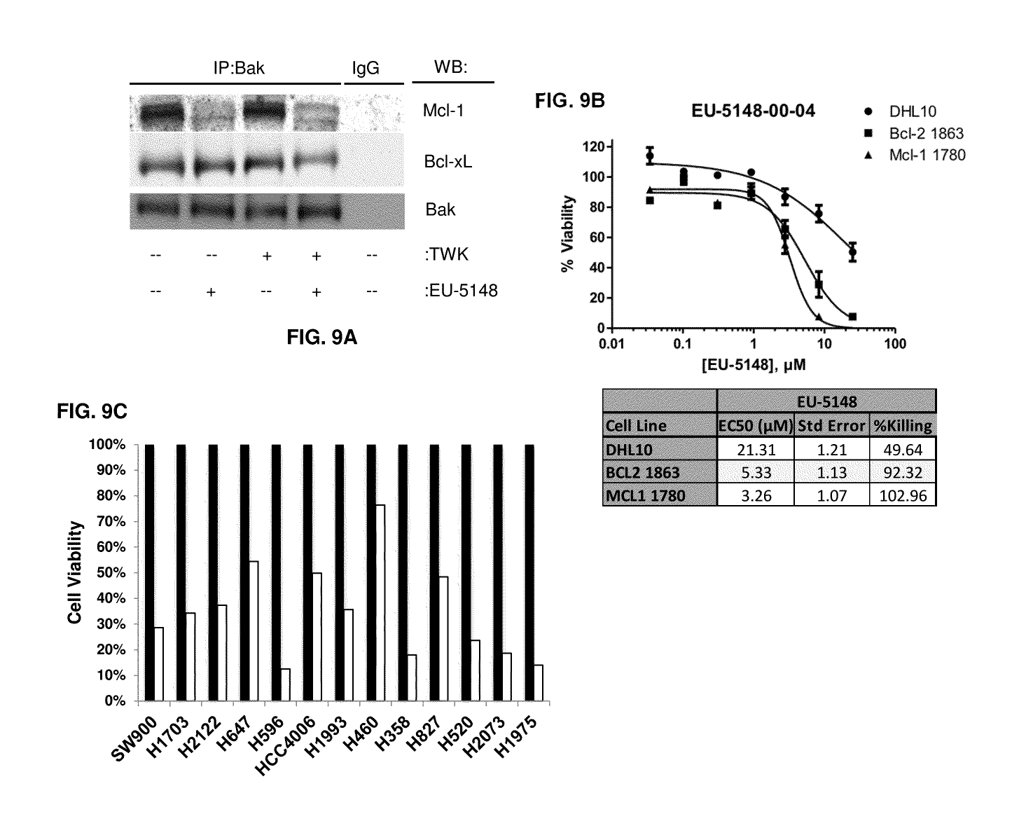

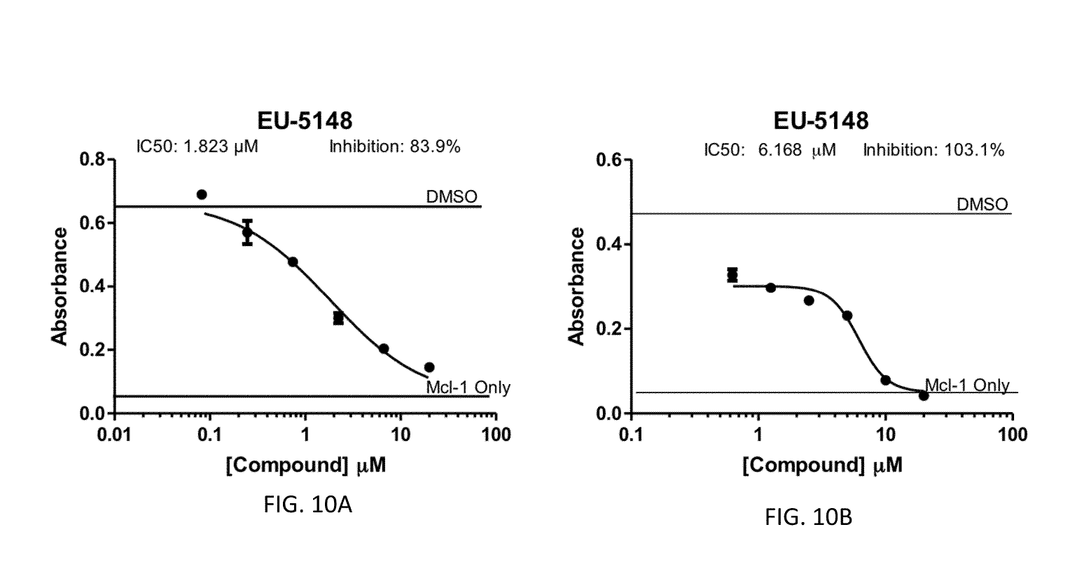

While inhibitors of Bcl-2/Bcl-xL have been well described, specific inhibitors of Mcl-1 are now being investigated. We explored the use of a Mcl-1-specific pharmacological inhibitor designated EU-5148 as an antagonist of NSCLC cell survival. FIG. 9A demonstrates that exposure to EU-5148 specifically disrupts the protein-protein interaction of Mcl-1 and Bak, while not affecting the interaction of Bcl-xL and Bak. H1975 cells were treated with EU-5148 in the presence or absence of TWEAK. Cells were immunoprecipitated with anti-Bak antibodies and immunoblotted for Mcl-1, Bcl-xL and Bak. Exposure to EU-5148 suppressed the protein interaction between Mcl-1 and Bak with or without TWEAK exposure. Conversely, exposure to EU5148 had no effect on the protein interaction of Bcl-xL and Bak. To further show Mcl-1 specificity for EU-5148, we employed cell lines deficient of Bax and Bim (DHL10) or driven by Mcl-1 (Mcl-1 1780) or Bcl-2 (Bcl-2 1863). FIG. 9B shows that cells driven by Mcl-1 are most sensitive to EU-5148 (EC50=3.26 μM), while cells deficient for Bax/Bim were less sensitive to EU-5148 (EC50=21.31 μM). Furthermore, an ELISA-based competitive displacement assay demonstrated that EU-5148 was ˜3.5-fold more disruptive of a Mcl-1-Bim protein interaction compared to a Bcl-xL-Bim protein interaction (FIGS. 10A and 10B).

In a panel of NSCLC cell lines, EU-5148 significantly diminishes cell viability 48 hours post-treatment compared to non-treated cells (FIG. 9C). Cell viability was reduced between 25-87% across 13 NSCLC cell lines with 11 of the 13 lines showing >50% reduction in cell viability.

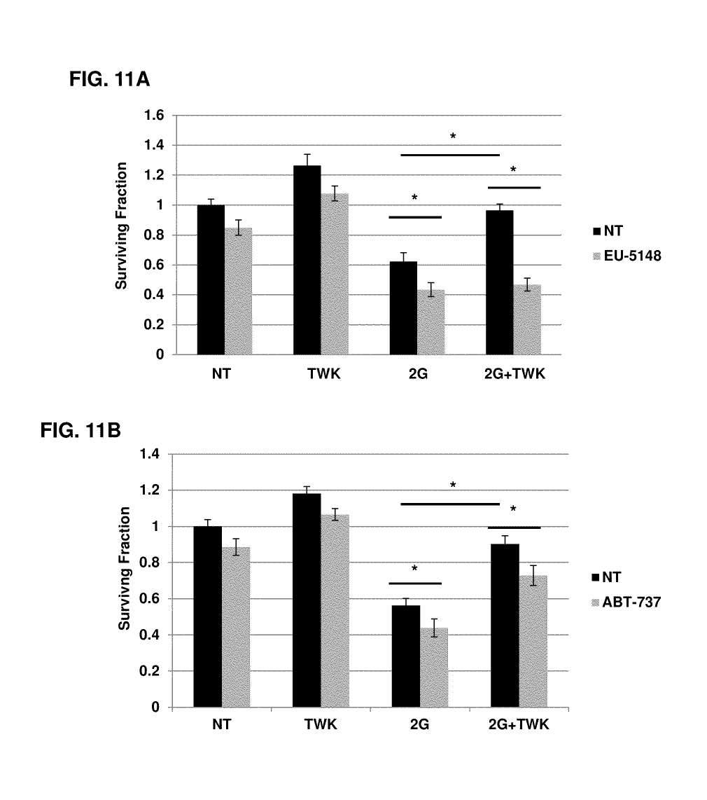

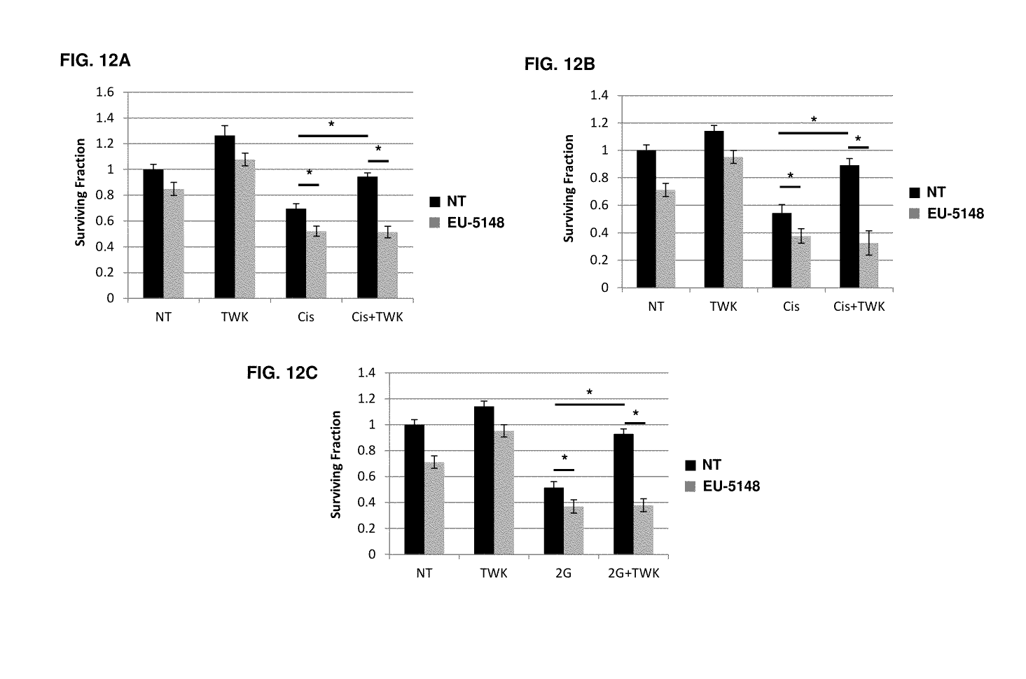

Lastly, we sought to assess whether EU-5148 could suppress TWEAK-induced NSCLC cell survival. Exposure of H1975 cells to radiation significantly reduces cell survival, whereas, exposure to the Mcl-1 inhibitor EU-5418 further enhances sensitivity to radiation (FIG. 11A). Pre-treatment with TWEAK completely abrogates the reduction in cell survival induced by radiation exposure, but Mcl-1 inhibition restores radiation sensitivity, even with TWEAK exposure. Similar effects are observed with EU-5418 exposure in H1975 cells exposed to cisplatin and H2073 cells exposed to radiation or cisplatin (FIGS. 12A-12C). While Mcl-1 inhibition abrogated TWEAK-induced cancer cell survival, exposure to ABT-737, a potent inhibitor of Bcl-2 and Bcl-xL, had a lesser effect on TWEAK-induced cell survival (FIG. 11B). Although ABT-737 did sensitize H1975 cells to radiation, the TWEAK-induced cell rescue was only minimally significant, suggesting a critical role for Mcl-1 in the TWEAK-induced cell survival effects in NSCLC.

It should be understood from the foregoing that, while particular embodiments have been illustrated and described, various modifications can be made thereto without departing from the spirit and scope of the invention as will be apparent to those skilled in the art. Such changes and modifications are within the scope and teachings of this invention as defined in the claims appended hereto.

What is claimed is:

1. A method of diagnosing cancer, the method comprising the steps of:
receiving a sample from a subject suspected of having cancer;
detecting a level of expression of Mcl-1 in the sample from the subject, wherein the level of expression of Mcl-1 is determined relative to a control sample;
detecting a level of expression of Fn14 in the sample from the subject, wherein the level of expression of Fn14 is determined relative to the control sample; and
wherein the subject is diagnosed as having cancer when the levels of expression of Mcl-1 and Fn14 are both elevated compared to the control sample.

2. The method of claim 1, wherein the cancer is lung cancer.

3. The method of claim 2, wherein the lung cancer is non-small cell lung cancer.

4. The method of claim 1 and further treating the cancer after diagnosis, the treatment comprising:
administering a therapeutically effective amount of a first pharmaceutical composition that inhibits Mcl-1 to the subject if the subject is diagnosed as having cancer.

5. The method of claim 4 and further comprising administering at least one of radiation and a second pharmaceutical composition to the subject.

6. The method of claim 5, wherein the second pharmaceutical composition comprises a therapeutically effective amount of a chemotherapeutic agent.

7. The method of claim 6, wherein the chemotherapeutic agent comprises cisplatin.

8. A method of predicting a cancer stage, the method comprising the steps of:
receiving a sample from a subject that has been diagnosed with cancer;
detecting a level of expression of Mcl-1 in the sample from the subject, wherein the level of expression of Mcl-1 is determined relative to a control sample;
detecting a level of expression of Fn14 in the sample from the subject, wherein the level of expression of Fn14 is determined relative to the control sample; and
wherein the cancer is determined to be at an advanced stage when the levels of Mcl-1 and Fn14 are both elevated compared to the control sample.

9. The method of claim 8, wherein the cancer is lung cancer.

10. The method of claim 9, wherein the lung cancer is non-small cell lung cancer.

11. The method of claim 8, wherein detection of expression of Mcl-1 and Fn14 in the sample is determined by the use of at least one of at least one antibody and at least one oligonucleotide.

12. A method of diagnosing and treating cancer, the method comprising the steps of:
diagnosing a subject as having cancer comprising the steps of:
receiving a sample from the subject suspected of having cancer;
adding a first reagent capable of binding to Fn14 to a mixture comprising the sample;
subjecting the mixture to conditions that allow detection of the binding of the first reagent;
diagnosing the subject as having cancer when the level of expression of Fn14 is elevated compared to a control sample; and
administering a therapeutically effective amount of a first pharmaceutical composition that inhibits Mcl-1 to the subject if the subject is diagnosed as having cancer.

13. The method of claim 12 and further comprising administering at least one of radiation and a second pharmaceutical composition to the subject.

14. The method of claim 13, wherein the second pharmaceutical composition comprises a therapeutically effective amount of a chemotherapeutic agent.

15. The method of claim 14, wherein the chemotherapeutic agent comprises cisplatin.

16. The method of claim 12, wherein the first pharmaceutical composition comprises small RNAs targeting SEQ ID NO: 1.

17. The method of claim 12, wherein the first pharmaceutical composition comprises pharmacological inhibitor.

18. The method of claim 12, wherein the cancer comprises lung cancer.

19. The method of claim 18, wherein the lung cancer comprises non-small cell lung cancer.

20. The method of claim 12 and further comprising the steps of:
adding a second reagent capable of binding to Mcl-1 to a mixture comprising the sample; and
subjecting the mixture to conditions that allow detection of the binding of the second reagent.
